(19) United States
(12) Patent Application Publication
(10) Pub. No.: US 2025/0359901 A1
(43) Pub. Date: Nov. 27, 2025

(54) INSTRUMENTS AND SURGICAL METHODS FOR BUNION PROCEDURES

(71) Applicant: Paragon 28, Inc., Englewood, CO (US)

(72) Inventors: Michael SCHMIDT, Greenwood Village, CO (US); Garrett Jeffrey LIPKER, Arvada, CO (US); Lucas Charles MCMAHAN, Lone Tree, CO (US); Kenneth Allan ROGGOW, Denver, CO (US); Dimitri PROTOPSALTIS, Memphis, TN (US)

(73) Assignee: Paragon 28, Inc., Englewood, CO (US)

(21) Appl. No.: 19/290,811

(22) Filed: Aug. 5, 2025

Related U.S. Application Data
(63) Continuation of application No. PCT/US2024/014953, filed on Feb. 8, 2024.
(60) Provisional application No. 63/484,092, filed on Feb. 9, 2023.
(60) Provisional application No. 63/579,717, filed on Aug. 30, 2023.

(51) Int. Cl. A61B 17/72; A61B 17/00; A61B 17/66

(52) U.S. Cl. CPC A61B 17/7291; A61B 17/66; A61B 2017/00477

(57) ABSTRACT

An alignment system having an alignment instrument with a lateral portion having an upper portion and lower portion, and a medial portion releasably couplable with the lateral portion and includes a distal portion and a medial portion. The distal portion and the medial portion are couplable with first and second bones and actuatable to apply compression between the bones. The alignment instrument includes a retention member couplable with the upper portion of the lateral portion of the alignment instrument and configured to engage with a third bone of the patient. A surgical method including the steps of coupling the retention member; coupling the lateral portion with the retention member; coupling the medial portion with the lateral portion; coupling the distal aspect of the medial portion; coupling the proximal aspect of the medial portion; applying compression between the second and third bones; and applying fixation across the second and third bones.

### CROSS REFERENCE TO RELATED APPLICATIONS

This application is a bypass continuation of International Patent Application No. PCT/US2024/014953, filed on Feb. 8, 2024, and entitled “Instruments and Surgical Methods for Bunion Procedures,” which claims priority benefit from U.S. Provisional Application No. 63/484,092 filed on Feb. 9, 2023, and entitled “Lapidus Clamp and Methods of Use,” and U.S. Provisional Application No. 63/579,717 filed on Aug. 30, 2023, and entitled “Instruments and Surgical Methods for Bunion Procedures,” the disclosures of each of these applications are hereby incorporated herein by reference in their entirety.

### TECHNICAL FIELD

The present disclosure relates to surgical instruments, guides, and methods of use to be implemented in surgical procedures. The present disclosure relates to podiatric and orthopedic surgical instruments, guides, and methodology to be implemented in various procedures of the foot and/or ankle, for example arthrodesis. More specifically, but not exclusively, the present disclosure relates to surgical instruments, guides to be implemented in conjunction with instruments (as well as other components, for example implants, devices, systems, assemblies, etc.) and methods of use for performing procedures to address bunions.

### BACKGROUND OF THE INVENTION

Many currently available surgical instruments and guides, as well as methodology, do not completely address the needs of patients. Additionally, many currently available surgical instruments, guides, and methodology fail to account for properties of joint anatomy and accordingly can decrease favorability of the outcome for the patient.

### SUMMARY OF THE INVENTION

The present disclosure is directed toward implants and implant systems for procedures involving the foot and/or ankle. More specifically, the present disclosure is directed to implants and implant systems for ankle procedures.

One aspect of the present disclosure is directed to an alignment system. The alignment system includes an alignment instrument, which includes a lateral portion having an upper portion and lower portion slidably adjustable and releasably couplable with one another, and a medial portion adjustable and releasably couplable with the lateral portion and having a distal portion and a medial portion. The distal portion and the medial portion are couplable with first and second bones of a patient and actuatable to apply a compression therebetween the first and second bones. The alignment system also includes a retention element releasably couplable with the upper portion of the lateral portion of the alignment instrument and configured to engage with a third bone of the patient.

Another aspect of the present disclosure is directed to a surgical method. The surgical method includes coupling a retention member with a first bone of a patient, coupling a lateral portion of an alignment instrument with the retention member, and coupling a medial portion of the alignment instrument with the lateral portion of the alignment instrument. The surgical method also includes coupling a distal aspect of the medial portion of the alignment instrument with a second bone of the patient, coupling a proximal aspect of the medial portion of the alignment instrument with a third bone of the patient, actuating the alignment instrument to apply compression between the second and third bones, and applying fixation across the second and third bones.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for the purpose of illustrating embodiments of inventions of the disclosure and are not to be construed as limiting the inventions.

FIG. 1 is a medial perspective view of an alignment system, in accordance with the present disclosure;

FIG. 2 is a lateral perspective view of the alignment system of FIG. 1, in accordance with the present disclosure;

FIG. 3 is a rear perspective view of the alignment system of FIG. 1, in accordance with the present disclosure;

FIG. 4 is a top view of the alignment system of FIG. 1, in accordance with the present disclosure;

FIG. 5 is a front view of the alignment system of FIG. 1, in accordance with the present disclosure;

FIG. 6 is a lateral view of the alignment system of FIG. 1, in accordance with the present disclosure;

FIG. 7 is a medial view of the alignment system of FIG. 1, in accordance with the present disclosure;

FIG. 8 is a front perspective view of a portion of the alignment system of FIG. 1, in accordance with the present disclosure;

FIG. 9 is a top view of the portion of FIG. 8 of the alignment system of FIG. 1, in accordance with the present disclosure;

FIG. 10 is a medial perspective view of a portion of the alignment system of FIG. 1, in accordance with the present disclosure;

FIG. 11 is a flow chart showing the process in which the alignment system of FIG. 1 may be implemented, in accordance with the present disclosure;

FIG. 12 is a medial perspective view of an alignment system, in accordance with the present disclosure;

FIG. 13 is a lateral perspective view of the alignment system of FIG. 12, in accordance with the present disclosure;

FIG. 14 is a top view of the alignment system of FIG. 12, in accordance with the present disclosure;

FIG. 15 is a top view of the alignment system of FIG. 12, in accordance with the present disclosure;

FIG. 16 is a front view of the alignment system of FIG. 12, in accordance with the present disclosure;

FIG. 17 is a lateral view of the alignment system of FIG. 12, in accordance with the present disclosure;

FIG. 18 is a medial view of the alignment system of FIG. 12, in accordance with the present disclosure;

FIG. 19 is a front perspective view of the alignment system of FIG. 12, in accordance with the present disclosure;

FIG. 20 is a top view of a portion of the alignment system of FIG. 12, in accordance with the present disclosure;

FIG. 21 is a lateral perspective view of a component of the alignment system of FIG. 12, in accordance with the present disclosure;

FIG. 22 is a top view of the component of FIG. 21 of the alignment system of FIG. 12, in accordance with the present disclosure;

FIG. 23 is a bottom view of the component of FIG. 21 of the alignment system of FIG. 12, in accordance with the present disclosure;

FIG. 24 is a rear view of the component of FIG. 21 of the alignment system of FIG. 12, in accordance with the present disclosure;

FIG. 25 is a front view of the component of FIG. 21 of the alignment system of FIG. 12, in accordance with the present disclosure;

FIG. 26 is a left side view of the component of FIG. 21 of the alignment system of FIG. 12, in accordance with the present disclosure;

FIG. 27 is a right side view of the component of FIG. 21 of the alignment system of FIG. 12, in accordance with the present disclosure;

FIG. 28 is a medial perspective view of a portion of the alignment system of FIG. 12, in accordance with the present disclosure;

FIG. 29 is an exploded perspective view of the portion of FIG. 28 of the alignment system of FIG. 12, in accordance with the present disclosure;

FIG. 30 is a perspective view of a resection instrument, in accordance with the present disclosure;

FIG. 31 is a top view of the resection instrument of FIG. 30, in accordance with the present disclosure;

FIG. 32 is a bottom view of the resection instrument of FIG. 30, in accordance with the present disclosure;

FIG. 33 is a right side view of the resection instrument of FIG. 30, in accordance with the present disclosure;

FIG. 34 is a left side view of the resection instrument of FIG. 30, in accordance with the present disclosure;

FIG. 35 is a front view of the resection instrument of FIG. 30, in accordance with the present disclosure;

FIG. 36 is a rear view of the resection instrument of FIG. 30, in accordance with the present disclosure;

FIG. 37 is a perspective view of a resection instrument, in accordance with the present disclosure;

FIG. 38 is a top view of the resection instrument of FIG. 37, in accordance with the present disclosure;

FIG. 39 is a bottom view of the resection instrument of FIG. 37, in accordance with the present disclosure;

FIG. 40 is a left side view of the resection instrument of FIG. 37, in accordance with the present disclosure;

FIG. 41 is a right side view of the resection instrument of FIG. 37, in accordance with the present disclosure;

FIG. 42 is a front view of the resection instrument of FIG. 37, in accordance with the present disclosure;

FIG. 43 is a rear view of the resection instrument of FIG. 37, in accordance with the present disclosure; and

FIG. 44 is a flow chart of the process in which the alignment system of FIG. 1 or FIG. 12 may be implemented, in accordance with the present disclosure.

### DETAILED DESCRIPTION OF THE INVENTION

In this detailed description and the following claims, the words proximal, distal, anterior, or plantar, posterior, or dorsal, medial, lateral, superior, and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation, and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

The instruments, implants, systems, assemblies, and related methods for maintaining, correcting, and/or resurfacing joint surfaces of the present disclosure may be similar to, such as include at least one feature or aspect of, the implants, systems, assemblies and related methods disclosed in International PCT Application No. PCT/US2018/20046, filed on Feb. 27, 2018, and entitled Intramedullary Nail Alignment Guides, Fixation Guides, Devices, Systems, and Methods of Use; International PCT Application No. PCT/US2018/64368, filed on Dec. 17, 2018, and entitled Alignment Guides, Cut Guides, Systems and Methods of Use and Assembly; International PCT Application No. PCT/US2019/041146, filed on Jul. 10, 2019, and entitled Guides, Instruments, Systems and Methods of Use; and/or International PCT Application No. PCT/US2014/27086, filed on Mar. 14, 2014, and entitled Intramedullary Nail Fixation Guides, Devices, and Methods of Use; and/or U.S. Pat. No. 9,980,760 filed on Nov. 19, 2014, and entitled Step Off Bone Plates, Systems, and Methods of Use; and/or U.S. Pat. No. D720,456 filed on Jul. 26, 2012 and entitled Lapidus Bone Wedge; and/or U.S. Pat. No. D765,844 filed on Oct. 23, 2014 and entitled Bone Plate; and/or U.S. Pat. No. D695,402 filed on Dec. 10, 2013 and entitled Lapidus Cut Guide; and/or U.S. Pat. No. D904,2016 filed on Nov. 22, 2017 and entitled Intramedullary Fastener; and/or U.S. Pat. No. D865,173 filed on Jul. 9, 2018 and entitled Cut Guide; and/or U.S. patent application Ser. No. 29/686,941 filed on Apr. 9, 2019 and entitled Cut Guide; and/or U.S. Pat. No. D904,609 filed on Apr. 9, 2019 and entitled Cut Guide; and/or U.S. Pat. No. D9042010 filed on Apr. 9, 2019 and entitled Cut Guide; which are hereby incorporated herein by reference in their entireties.

Similarly, the instruments, implants, systems, assemblies, and related methods for maintaining, correcting, and/or resurfacing joint surfaces of the present disclosure may include one or more instrument (e.g., one or more insertion and/or implantation instruments) disclosed in United Stated Provisional Application No. 63/173,043, filed Apr. 9, 2021 and entitled Surgical Instruments, Guides, and Methods of Use; and/or International PCT Application No. PCT/US2018/20046, filed on Feb. 27, 2018, and entitled Intramedullary Nail Alignment Guides, Fixation Guides, Devices, Systems, and Methods of Use; and/or International PCT Application No. PCT/US2018/64368, filed on Dec. 17, 2018, and entitled Alignment Guides, Cut Guides, Systems and Methods of Use and Assembly; and/or International PCT Application No. PCT/US2019/041146, filed on Jul. 10, 2019, and entitled Guides, Instruments, Systems and Methods of Use; and/or International PCT Application No. PCT/US2014/27086, filed on Mar. 14, 2014, and entitled Intramedullary Nail Fixation Guides, Devices, and Methods of Use; and/or U.S. Pat. No. 9,980,760 filed on Nov. 19, 2014, and entitled Step Off Bone Plates, Systems, and Methods of Use; and/or U.S. Pat. No. D720,456 filed on Jul. 26, 2012 and entitled Lapidus Bone Wedge; and/or U.S. Pat. No. D765,844 filed on Oct. 23, 2014 and entitled Bone Plate; and/or U.S. Pat. No. D695,402 filed on Dec. 10, 2013 and entitled Lapidus Cut Guide; and/or U.S. Pat. No. D904,2016 filed on Nov. 22, 2017 and entitled Intramedullary Fastener; and/or U.S. Pat. No. D865,173 filed on Jul. 9, 2018 and entitled Cut Guide; and/or U.S. patent application Ser. No. 29/686,941 filed on Apr. 9, 2019 and entitled Cut Guide; and/or U.S. Pat. No. D904,609 filed on Apr. 9, 2019 and entitled Cut Guide; and/or U.S. Pat. No. D9042010 filed on Apr. 9, 2019 and entitled Cut Guide; and/or U.S. Provisional Patent Application No. 63/262,845 filed on Oct. 21, 2021 and entitled Surgical Instruments, Guides, and Methods of Use; and/or U.S. Provisional Patent Application No. 63/304,144 filed on Jan. 28, 2022 and entitled Surgical Instruments, Guides, and Methods of Use; and/or U.S. Provisional Patent Application No. 63/484,092 filed on Feb. 9, 2023 and entitled Lapidus Clamp and Methods of Use; which are hereby incorporated herein by reference in their entireties.

Procedures to address deformities such as bunions and anatomical structures of and around the Lapidus joint frequently require the positioning/repositioning and/or rotation/derotation of the first metatarsal. Referred to herein as the “Lapidus” joint, this joint may also be known and referred to as the first tarsometatarsal joint. It is common for a procedure of the Lapidus joint (e.g., fusion/arthrodesis) to require that the first metatarsal be manipulated by applying one or more forces to the first metatarsal. In some procedures, this manipulation is necessary before any cutting and/or preparation and subsequent fusion of the Lapidus joint can take place. In evaluating a Lapidus joint deformity, two different criteria are typically analyzed for correction. One of these criteria is the intramedullary angle formed between the longitudinal axes of the first metatarsal and the second metatarsal. Bunion deformities and other conditions of the Lapidus joint often include the first metatarsal shifting medially from a normal anatomical position, thus increasing the IM angle between the first and second metatarsals from what can be considered an anatomically correct range of angle measures. Rotation of the first metatarsal is also analyzed, as bunion deformities and other conditions of the Lapidus joint commonly include a first metatarsal that has rotated substantially in the frontal plane in a substantially clockwise direction (when viewed from an anterior to posterior direction). Commonly, a Lapidus joint procedure such as those mentioned previously requires manipulation of the first metatarsal so as to a) correct (e.g., decrease) the IM angle between the first and second metatarsals by applying a substantially lateral force to the first metatarsal; and/or b) derotate the first metatarsal which as rotated from a normal anatomical position by applying a rotational force in a substantially counterclockwise direction when the first metatarsal is viewed in an anterior to posterior direction.

Referring to the drawings included herein, instrument systems and associated methods are shown and described. It should be understood that one or more of the instrument systems and/or associated methods shown and described herein may be implemented in conjunction with one or more of the other various instrument systems, components thereof, and associated methods shown and described herein. Further, it should be understood that the instrument systems and methods shown herein-as well as components thereof-may be duplicated, eliminated, or otherwise combined/modified and incorporated in conjunction with the same or other systems including but not limited to those shown and described herein and those incorporated by reference previously herein.

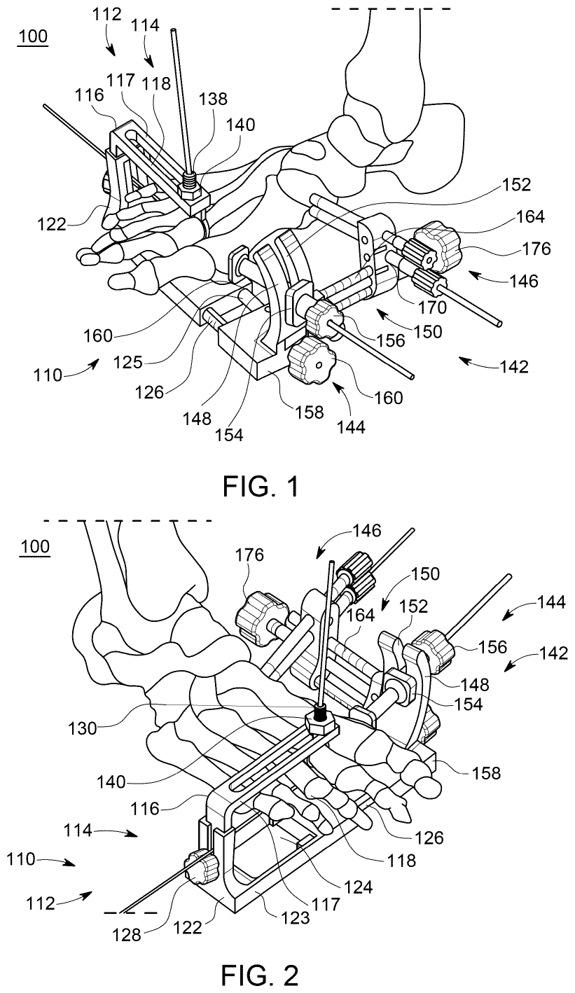

Referring now to FIGS. 1-10, an alignment instrument system 100 (referred to hereinafter as “instrument system 100” or “system 100”) is shown, according to an exemplary embodiment. The system 100 may be implemented to aid a physician in positioning, repositioning, or referencing various anatomy of a patient intraoperatively, particularly bony structures of the foot including, for example, a first metatarsal 202, a medial cuneiform 204, and a second metatarsal 206 of a patient. It should be understood, however, that implementation of the system 100 may include the system interfacing, releasably coupling, referencing, manipulating, or otherwise incorporating various structures of the foot or ankle of a patient including but not limited to the first metatarsal 202, the medial cuneiform 204, and the second metatarsal 206. Additionally, the system 100 may be implemented in conjunction with one or more systems (e.g., instrument systems, implant systems, etc.) and/or components thereof (e.g., implants, instruments, etc.) including but not limited to those incorporated by reference herein. Further, the system 100 may also be implemented in conjunction with various surgical methodology, for example for bunion procedures or other procedures to address anatomy of the midfoot and forefoot. As such, the system 100 may be implemented in conjunction with the surgical method shown and described subsequently herein, as well as other methodology.

The system 100 is shown to include an alignment instrument 110 (referred to hereinafter as “instrument 110”) and a retention member 130, with the retention member 130 releasably couplable with the instrument 110. The system 100 may also include various stabilization wires, also referred to as “k-wires” configured to facilitate coupling between one or more components of the system 100 (for example, the instrument 110 and the retention member 130) and bony anatomy of a patient (for example, the first metatarsal 202, the medial cuneiform 204, and the second metatarsal 206). As shown, the instrument 110 may have a substantially clamp-like geometry (e.g., opposing elements configured to be manipulated in order to apply a biasing force therebetween) and accordingly, clamp-like functionality (e.g., configured to be actuated in order to reposition or retain in a position one or more components with which the instrument interfaces). The instrument 110 may include one or more features configured to directly couple with the anatomy (e.g., interface with, contact, or otherwise address the anatomy) and/or may include one or more features configured to indirectly couple with the anatomy (e.g., by coupling with an intermediate component, which in turn couples with the anatomy).

The instrument 110 is shown to include a lateral portion 112 and a medial portion 142, where the lateral and medial portions 112, 142 are positioned substantially opposite the instrument 110 from one another. As shown in FIGS. 1-10, the lateral and medial portions 112, 142 may be releasably and translatably coupled with one another such that one or both components may be adjusted (e.g., translated in a single plane or along an axis) relative to the other. Further, the instrument 110 may be assembled and disassembled via the releasable coupling of the lateral and medial portions 112, 142.

The lateral portion 112 is shown to include a body 114 including an upper portion 116 substantially opposite the body 114 from a lower portion 122. The upper portion 116 may have a substantially L-shaped geometry (e.g., a geometry having one approximately 90-degree angle), and include an arm 117 extending medially (as shown in the configuration of FIGS. 1-10) from the angle. The arm 117 includes a cavity 118 therein and extending therethrough from a top surface of the arm 117 to a bottom surface and having a substantially oblong, elongated geometry. In some aspects, the arm 117 may include multiple cavities 118 disposed variously along the length of the arm 117. In the previous example, the multiple cavities 118 may be variously shaped and further may collectively have a similar footprint and lateral dimension to the cavity 118 as shown in FIGS. 1-9. The arm 117 also includes an opening 119 disposed at a terminal end of the arm 117, where the opening 119 has as substantially circular geometry and extends through the arm 117 from the top surface through to the bottom surface (similar to the cavity). The opening 119 may be configured to facilitate releasable coupling with the retention member 130, where at least a portion of the retention member 130 is received at least partially therein and/or therethrough the opening 119. The upper portion 116 further includes a plurality of openings 120 disposed opposite the approximately 90-degree angle from the arm 117. As shown in at least FIG. 6, the openings 120 may be arranged vertically and have equal dimensions and vertical spacing therebetween. Further, the openings 120 may also include a larger opening than those having equal size and vertical spacing, with the larger opening positioned below the equally sized openings and configured to receive at least a portion of a fastener 128 therein and/or therethrough so as to facilitate coupling of the upper and lower portions 116, 122 of the body 114. Further, the larger opening of the openings 120 may also including a threading on an inner portion thereof (e.g., to interface with a complementary threading of the fastener 128) and be configured to, in conjunction with the fastener 128, retain the upper portion 116 at a desired height as the upper portion 116 extends from a portion of the lower portion 122. Accordingly, actuation of the fastener 128 may permit vertical translation of the upper portion 116 relative to the lower portion 122 and/or retain the upper portion 112 in a desired position (e.g., at a desired height) relative to the lower portion 116. Such actuation of the fastener 128 and/or the upper and lower portions 112, 116 may be performed in order to adjust a height of the lateral portion according to a size and height of a forefoot and/or midfoot of a patient to achieve a position the same or similar to that as shown in at least FIGS. 1-6.

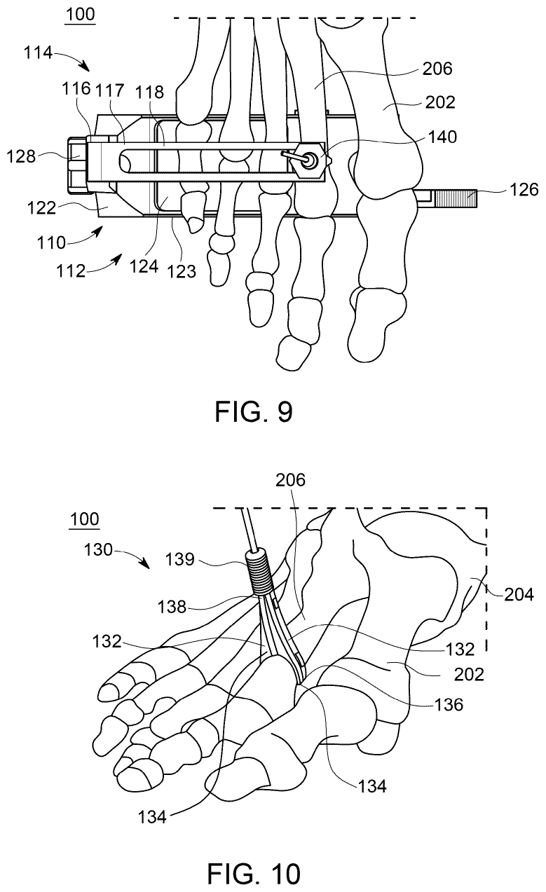

The lower portion 122 of the body 114 is shown to have a similar geometry to that of the upper portion 116 in that the lower portion 122 has a geometry that includes an approximate right angle, according to the exemplary embodiment of FIGS. 1-9. The lower portion 122 as shown is configured to receive at least a portion of the upper portion 116 therein so as to facilitate the aforementioned adjustment and translation therebetween. The lower portion 122, as shown in FIG. 6, includes an at least partially open feature on a lateral-most portion thereof so as to facilitate placement of the fastener 128 within the largest of the openings 120, as well as any desired placement of a k-wire through one or more of the remaining openings 120. Opposite the approximately 90-degree angle from the at least partially open feature, the lower portion 122 includes an arm 123 extending in a plane that, when coupled with the upper portion 116, is substantially parallel to the plane in which the arm 117 extends. Further, the arm 123 extends laterally and as shown in FIG. 9, the footprint of the arm 117 is substantially centered within the footprint of the arm 123. The arm 123 is also shown to include a cavity 124 disposed therein and extending at least partially along a length thereof. Similar to the cavity 118, in some aspects the cavity 124 may include multiple cavities 124 which may occupy a substantially similar length of the arm 123 to the single cavity 124 as shown at least FIGS. 8-9.

The lower portion 122 is further shown to include an opening 125 and a coupling member 126 disposed at a terminal end of the arm 123. The coupling member 126, as shown, may be threadably coupled in an opening the same as or similar to the opening 125. Further, the coupling member 126 may be configured to releasably and threadably couple with a complementary coupling feature of the medial portion 142 so as to facilitate the aforementioned releasably coupling and translatability between the lateral and medial portions 112, 142. The opening 125 may be configured to receive (at least partially therethrough/therein) and releasably and threadably couple with a component the same as or similar to the coupling member 126, for example a coupling member of the medial portion 142. Similarly, the coupling member 126 and a complementary component of the medial portion may, collectively, facilitate both releasably coupling and adjustable translation between the lateral and medial portion 112, 142. For example, the coupling member 126, the opening 125, and complementary components of the medial portion 142 may facilitate adjustment of the instrument 110 in the medial-lateral direction such that the instrument 110 may have an adjustable width to accommodate various sizes/widths of feet of the patient. The lower portion 122 is further shown to include an actuator 127, which is shown in at least FIG. 6 as a button/switch mechanism. The actuator 127 may be configured to facilitate the releasable coupling of the lateral and medial portions 112, 142 with respect to the opening 125, the coupling member 126, and other complementary components of the medial portion 142. For example, the actuator 127 may be manipulatable from a first, locking/retaining position in which decoupling or translation of the lateral and/or medial portions 112, 142 is not permitted to a second, unlocked position is which decoupling or translating the lateral and medial portions 112, 142 is permitted (e.g., the aforementioned adjustment may occur).

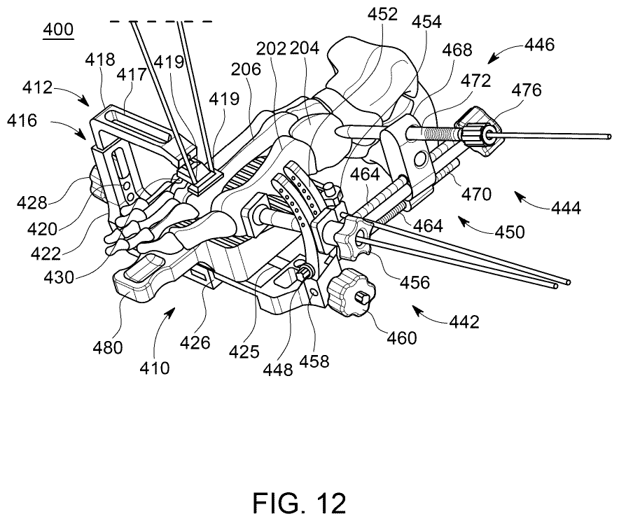

As shown in FIGS. 1-10, and with particular reference to FIG. 10, the retention element 130 (e.g., the metatarsal grip 130) is shown in a decoupled state from the clamp portion 110. The retention element 130 is shown to include a shaft 138 extending in a direction opposite (e.g., proximally, with the pair of projections 132 extending distally) the pair of projections 132. The shaft 138 is configured to be integral with each of the pair of projections 132 and, as shown in FIG. 12, has a substantially cylindrical geometry. The shaft 138 is shown to include a circumferential threading 139 extending along the entirety of the shaft 138 (although in some embodiments, the threading 139 may extend along at least a portion of the shaft 138). As shown in FIG. 8, at least a portion of the shaft 138 and threading 139 thereof is configured to be received therein and therethrough the opening 119 such that a coupling element 140 may be releasably and threadably coupled with the threading 139 at a position superior relative to the arm 117, thus coupling the retention member 130 with the lateral portion 112. Each of the pair of projections 130 is shown to extend from the shaft 138 at a substantially perpendicular angle (or lack of angle) along an interior portion thereof, and are further shown to extend from the shaft at a substantially oblique angle from one another along an exterior portion thereof (where the angles or lack of angles are based on an extended plane positioned on the surface of the interior portions and exterior portions of the projections 132, respectively). Each of the pair of projections 132, shown in FIG. 12 as a pair of prongs or other extension elements (protrusions, arms, etc.) extend from the shaft 138 adjacent to the distal-most portion of the threading 139.

As shown, each of the pair of projections 132 include a lobe 134 extending from the inner surface of each of the projections 132 such that a first portion of the lobe 134 includes a straight edge parallel to an opposing parallel edge of the opposing lobe 134 (e.g., substantially parallel to a longitudinal axis of the shaft 138). Similarly, each of the lobes 134 includes a second portion with a second straight edge substantially perpendicular to the first straight edge of each lobe 134. In some aspects, one or both of the lobes 134 may contact a superior (e.g., upper) portion of a metatarsal (e.g., the second metatarsal) such that the retention element 130 contacts the medial and lateral surfaces of the second metatarsal via the distal portion of the pair of projections 132, and the superior surface of the second metatarsal by at least a portion of one or both of the lobes 134. In some aspects the lobes 134 may have alternate geometries, for example hemispherical or another alternative geometry. Each projection 132 of the pair of projections 132 is shown to include a texture 136 disposed on an interior portion thereof and arranged distally relative to the pair of lobes 134. In some aspects, the texture 136 may be uniform on both of the pair of projections 132, or may vary from one single projection of the pair of projections 132 to the other. Further, the texture 136 may be configured such that contact of the texture 136 with the medial and lateral surfaces of the second metatarsal may create friction, thus increasing the retention of the second metatarsal between the pair of projections 132.

The medial portion 142 includes a distal portion 144 substantially opposite the medial portion 142 from a proximal portion 146, according to an exemplary embodiment. The distal portion 144 may be releasably and translatably coupled with the proximal portion 146 via one or more coupling members and/or mechanisms, which may be the same as or similar to those facilitating the releasable and translatable coupling between the lateral and medial portions 112, 142.

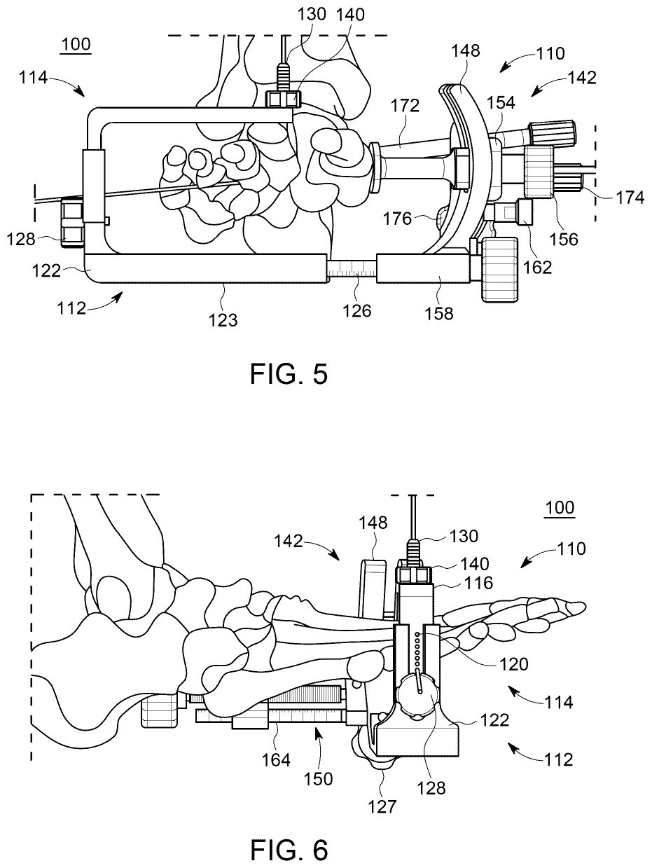

As shown in at least FIGS. 5-7, the distal portion 144 includes an upper portion 148 and a lower portion 158. As shown, the upper and lower portions 148, 158 are integral with one another but in some aspects may be releasably couplable with one another. The upper portion 148 may have a substantially curved geometry as it extends upward from the lower portion 158, where the curvature extends outward medially before curving laterally (e.g., a convex geometry facing medially and a concave geometry facing laterally). Further, the upper portion 148 may be positioned as a substantially oblique angle relative to a longitudinal axis of the medial portion 142 that extends substantially in the anterior-posterior direction. The upper portion 148 is shown to include a slot 152 extending vertically along an upper portion thereof and, as shown, is open on its upper-most edge so as to facilitation insertion and/or removal of a component into the slot 152. As shown, the slot 152 defines a plane (or series of planes) extending therethrough that forms a substantially oblique angle with the coupling member 126 and plane or longitudinal axis thereof.

The upper portion 148 includes a slider 154 positioned at least partially within the slot 152 and having a geometry along at least a portion of a length thereof having a lateral dimension less than the width of the slot 152. Accordingly, the slider 154 may be positioned at least partially within the slot 152 and translated in a substantially vertical direction along an arcuate path defined by the aforementioned geometry of the upper portion 148. As shown, the slider 154 may include a variety of features and/or components along a length thereof, which may also include various lateral dimensions. For example, on opposite sides of the portion of the slider 154 with a lateral geometry lesser than that of the slot 152, the slider 154 may include geometric features having a lateral dimension greater than that of the slot, so as to retain the slider 154 within the slot 154 (from a medial-lateral perspective). Further, the slider 154 may be configured to be cannulated so as to receive a component therein and at least partially therethrough (for example, a k-wire) the cannulation, which may extend along a longitudinal axis of the slider 154. The slider 154 may also include one or more surfaces, for example at a terminal end, with a contoured geometry and/or surface configured to facilitate interfacing with a bony anatomy of a patient (for example, the first metatarsal 202). The slider 154 is also shown to include an actuator 156 disposed on a medial-most portion of the slider 154 (as shown in FIG. 5). The actuator 156 may also include a cannulation configured to align with the cannulation of the slider 154 such that a single k-wire may be received into and through both components (for example, to releasably couple the slider 154 and actuator 156 with the first metatarsal 202 and facilitate manipulation of the first metatarsal 202 via actuation of the slider 154 along the path defined by the slot 152). The actuator 156 may be manipulatable from a first position in which the slider 154 is translatable along the arcuate path (which, in some embodiments, may be a substantially vertical path) defined by the slot 152 of the upper portion 148 to a second position in which the slider 154 is retained in a desired position along the arcuate path of the slot 152.

The lower portion 158 is shown to include a coupling member 160 which is shown to extend through and opening in the lower portion 158 from the medial side to the lateral side, as shown in at least FIGS. 4-5. In some aspects, the coupling member 160 may include one or more features the same as and/or similar to those of the coupling member 126 as shown and described previously. As shown, the coupling member 160 is positioned posterior relative to the coupling member 126 and, further, is configured in a substantially parallel orientation to the coupling member 126. The coupling member 160 may include an actuator (shown as a knob) which is positioned on the medial side of the lower portion 158 while an elongated portion of the coupling member 160 (which may include a threading) extends into and through the lower portion 158 to be received at least partially within the opening 125 of the lateral portion 112 so as to facilitate coupling between the lateral and medial portions 112, 142. Rotation of the knob of the coupling member 160 may facilitate engagement/disengagement of the coupling member 160 with threading of openings in the lower portion 158 and the lateral portion 112. Similarly, the lower portion 158 may include an opening the same as and/or similar to the opening 125 that is configured to receive at least a portion of the coupling member 126 therein. Collectively, the aforementioned openings and coupling members 126, 160 are configured to facilitate coupling between the lateral and medial portions 112, 142 as well as translatable adjustment in the medial-lateral direction to accommodate a size and/or width of a foot of a patient. In some embodiments, the coupling members 126, 160 may both be coupled with either the lateral portion 112 or the medial portion 142 prior to coupling with the complementary component. Further, one or more of the coupling members 126, 160 may be removable from the lateral and medial components 112, 142 entirely so as to facilitate disassembly of the instrument 110.

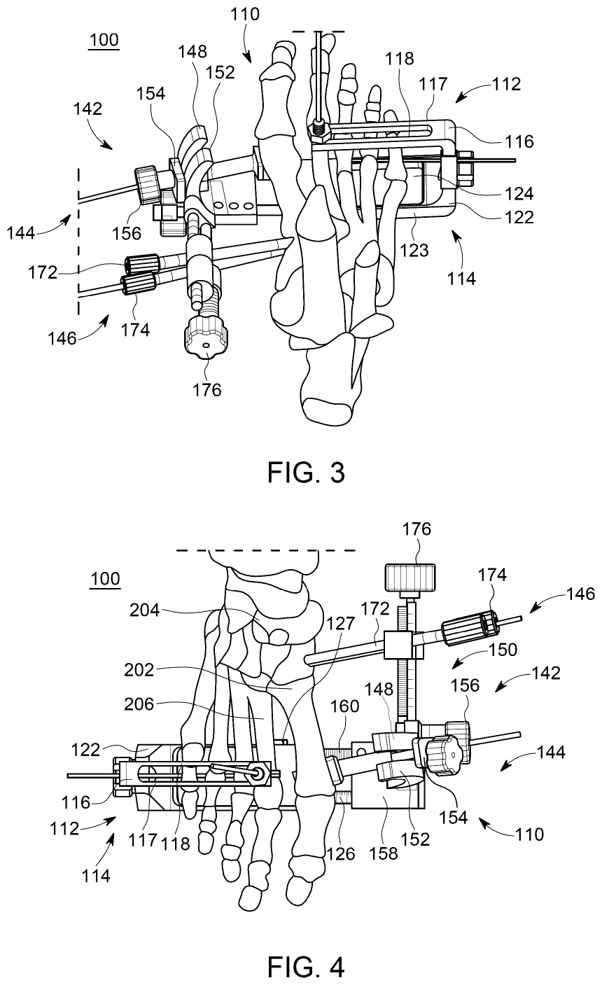

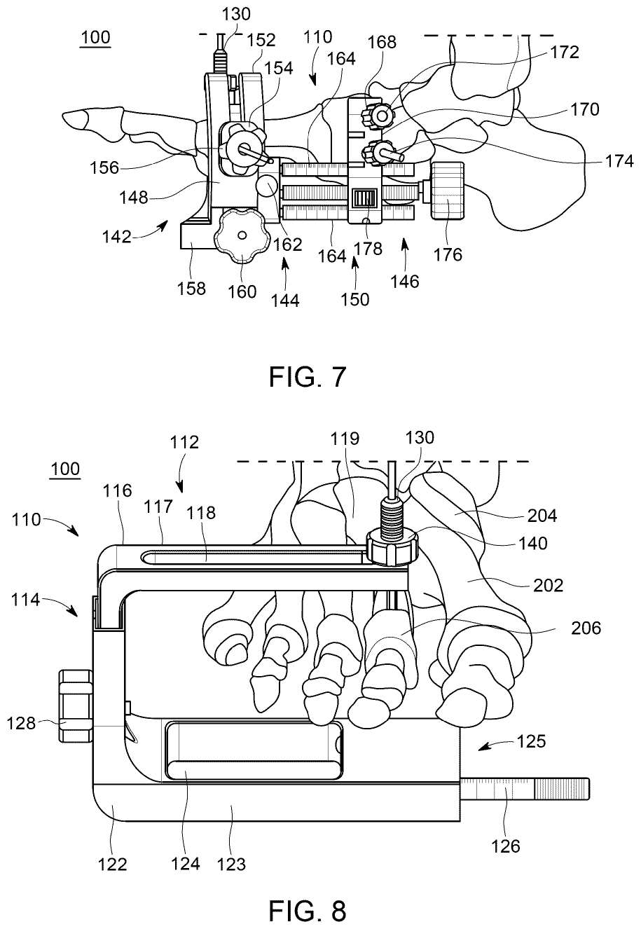

As shown in at least FIG. 3, the proximal portion 146 has a similarly curved geometry to that of the upper portion 148 of the distal portion 144 (although the proximal portion 146 may include an alternate or more gradual arcuate shape). The proximal portion 146, which is shown to be coupled with the distal portion 144, is shown to include an upper opening 168 and a lower opening 170 extending through the proximal portion 146 from a medial surface to a lateral surface. As shown, the openings 168, 170 include a substantially circular and cylindrical geometry so as to receive at least a portion of at least one of an upper guide 172 and/or a lower guide 174 therein and therethrough. Each of the guides 172, 174 may be interchangeable in that each guide may have compatibility with each of the openings 168, 170. Further, each of the guides 172, 174 may include a cannulation along a longitudinal axis thereof configured to receive a coupling element, for example a k-wire, therein and therethrough (for example, to facilitate coupling of the proximal portion 146 with the medial cuneiform 204). As shown in FIG. 7, the upper opening 168 includes an indication of “nail” adjacent to the opening 168 on a medial surface of the proximal portion 146, whereas the lower opening 170 includes an indication of “plate” adjacent to the opening 170 on a medial surface of the proximal portion 146. The aforementioned indications may be configured to guide optimal placement of a k-wire through one of the openings 168, 170 (via guide 172 or 174) and into the medial cuneiform 204 so as to couple the proximal portion 146 with the medial cuneiform 204 and also avoid any space needed (e.g., surfaces of anatomy or areas above the foot of the patient) to implement systems for applying fixation across the first tarsometatarsal joint (for example, a plate or nail system, where a physician would place the aforementioned k-wire in the opening 168, 170 corresponding to the desired fixation means).

The distal portion 144 is slidably (e.g., translatably) and threadably coupled with the proximal portion 146 and, as shown in at least FIG. 7, are coupled via a compression mechanism 150 disposed therebetween. The compression mechanism 150 is shown to include a pair of coupling elements 164, shown in FIG. 7 as elongated threaded members, configured to threadably engage and couple with the distal and proximal portions 144, 146 via openings on side surfaces thereof which may be the same as and/or similar to the opening 125 as shown and described previously. As shown, the coupling elements 146 are positioned substantially parallel to one another and in a co-planar configuration. In some aspects, the coupling elements 164 may extend into and through the proximal portion 146 as shown in FIG. 7. The coupling elements 164 may be configured to facilitate adjustment of the medial portion 142 and the distal and proximal portions 144, 146 thereof such that a physician may adjust the medial portion 142 of the instrument 110 according to a size and/or length of a foot of a patient. Further, the compression mechanism 150 includes a coupling member 176 positioned vertically between the coupling elements 164 and engaging with the distal and proximal portions 144, 146 via the same or similar openings disposed on side surfaces of the distal and proximal portions 144, 146. The coupling member 176 may be the same as or similar to the coupling member 160 and include a knob portion as well as an elongated portion extending from the knob and having a threading along at least a portion thereof. Actuation of the knob of the coupling member 176 may be configured to engage the threading thereof with complementary threading of at least one of the distal and proximal portions 144, 146. For example, if the distal portion 144 is coupled with the first metatarsal 202 of a patient and the proximal portion 146 is coupled with the medial cuneiform 204 of the patient, rotation of the knob of the coupling member 176 is configured to apply a compressive (or, if rotated in the opposite direction, a distractive) force across the first tarsometatarsal joint so as to bias the first metatarsal 202 and the medial cuneiform 204 (which may include resected surfaces thereof) toward one another. The distal portion 144 is shown to include a locking button 162, which may be configured to retain the medial portion 142 in the aforementioned compressive state when engaged by a physician (e.g., so fixation can be applied with the first metatarsal 202 and medial cuneiform 204 are being biased toward one another). The proximal portion 146 is also shown to include an actuator 178, shown in FIG. 7 as a button or switch mechanism which, when actuated, may enable disassembly of the medial portion 142 and components thereof.

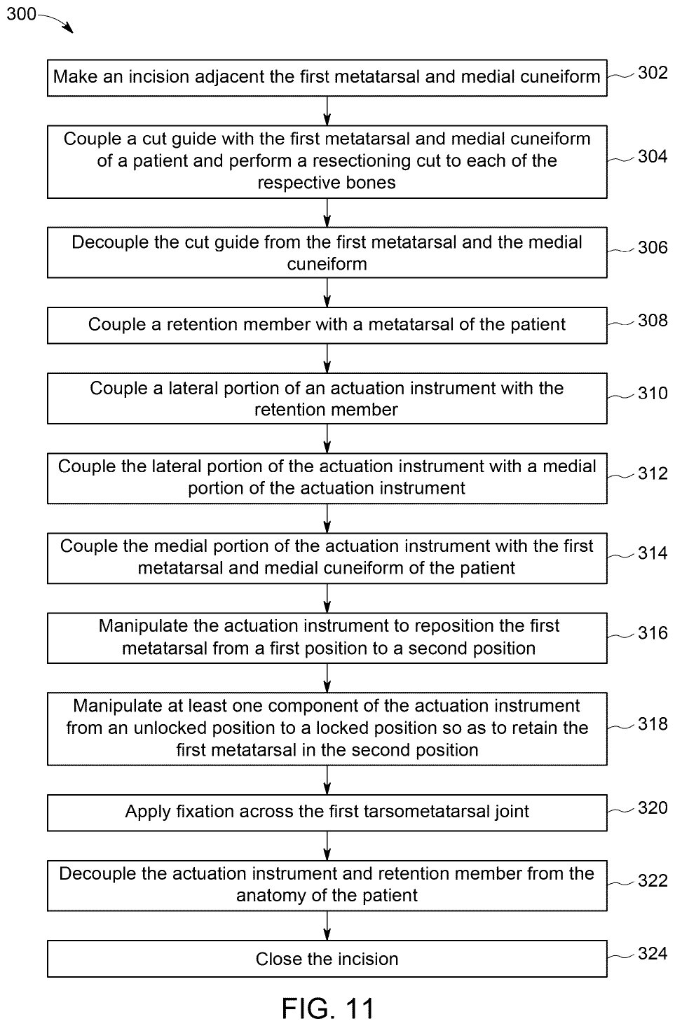

Referring now to FIG. 11, a process 300 for performing at least a portion of a bunion procedure is shown, according to an exemplary embodiment. In performing process 300, the system 100 and/or one or more components thereof (in addition to other systems/components) may be implemented. Further, it should be understood that in performing process 300, one or more of the steps thereof may be omitted, repeated, performed in an alternate sequence, or replaced with one or more alternate steps.

Process 300 is shown to include a step 302 of making an incision adjacent to the first metatarsal and the medial cuneiform, according to an exemplary embodiment. In some aspects, one or more components of the system 100 may be adjacent to anatomy of the patient when a physician performs step 302. Further, the incision made in step 302 may be positioned so as to facilitate placement and coupling of a specific cut guide (including, for example, those incorporated by reference herein) with/adjacent to the first tarsometatarsal joint (e.g., to couple a first portion of the guide with the first metatarsal 202 and a second portion of the guide with the medial cuneiform 204).

Process 300 is shown to include a step 304 of coupling a first cut guide with the first metatarsal of a patient and performing a resection cut, according to an exemplary embodiment. Step 304 may include implementing a cut guide that is not couplable with other components of the system 100, for example one or more of the cut guides incorporated by reference herein. In some aspects, the cut guide may be coupled with the first metatarsal 202 and the medial cuneiform 204 via k-wires and the resection cuts made with a sagittal or reciprocating saw so as to create a flat surface that is not slanted in any of the anterior/posterior/medial/lateral directions. Further, the cut guide may be configured to guide the cuts to the first metatarsal 202 and the medial cuneiform 204 to a desired angle or obliquity (e.g., configure the flat surfaces resulting from the cuts to have a known angle between one another).

Process 300 is shown to include a step 306 of decoupling the first cut guide from the first metatarsal, according to an exemplary embodiment. As mentioned in step 304, the cut guide may be coupled with the first metatarsal 202 via a k-wire and, accordingly, step 304 may include removing the cut guide over the k-wire or removing the k-wire. Similarly, step 306 may also include removing a k-wire from the medial cuneiform 204 in order to remove the cut guide, or removing the cut guide over the k-wire placed in the medial cuneiform 204. Step 306 may also include removing any debris created by and remaining from the resection cuts made in step 304, as some such debris may not be accessible without removal of the cut guide.

Process 300 is shown to include a step 308 of coupling retention member with a metatarsal of the patient, according to an exemplary embodiment. The retention member of step 308 may be the same as or similar to the retention member 130 as shown and described previously. Further, as shown in FIG. 1-10, the retention member 130 is shown to be releasably coupled with the second metatarsal 206 of the patient via a k-wire placed in the second metatarsal 206 and extending upward in a superior direction such that at least a portion of the k-wire is disposed within the cannulation of the retention member so as to facilitate coupling with the second metatarsal 206. When coupled with a metatarsal of a patient, for example the second metatarsal 206, the retention member is positioned such that the legs of the retention member substantially straddle and retain at least partially there between the metatarsal of the patient.

Process 300 is shown to include a step 310 of coupling a lateral portion of an actuation instrument with the retention member, according to an exemplary embodiment. Step 310 may include manipulating the lateral portion 112 of the instrument 110 such that at least a portion of the shaft 138 of the retention member 130 is received within the opening 119 of the arm 117 of the upper portion 116 of the lateral portion 112. Further, step 310 may include threadably coupling the coupling element 140, which may be a threaded nut, bolt, or other fastening-like component with the threading 139 of the retention member 130 in a position superior relative to the arm 117. In some aspects, step 310 may also include adjustment of the height of the lateral portion 112 by manipulating the upper portion 116 relative to the lower portion 122 and, further, manipulating the fastener 128 thereof so as to secure the upper portion 116 relative to the lower portion 122 at the desired height. In some aspects, step 310 may also include placement of a k-wire through one or more of the openings 120, which may further couple with a bony anatomy of a patient.

Process is shown to include a step 312 of coupling the lateral portion of the actuation instrument with a medial portion of the actuation instrument, according to an exemplary embodiment. Step 312 may include manipulating and/or actuating one or more of the coupling members 126, 160 so as to guide at least a portion of the coupling elements into the opening 125 and/or other similar openings disposed on the lateral and medial portions 112, 142. In some aspects, step 312 may include adjusting the distance between the lateral and medial components 112, 142 according to a size/width of a foot of the patient. The coupling members 126, 160 may further be manipulated in order to secure the lateral and medial portions 112, 142 in their desired positions relative to one another.

Process 300 is shown to include a step 314 of coupling the medial portion of the actuation instrument with the first metatarsal and medial cuneiform of the patient, according to an exemplary embodiment. Step 314 may include the placement of a k-wire through the cannulation of the slider 154 and the actuator 156 into the first metatarsal 202 of the patient, thus coupling the distal portion 144 of the medial portion 142 of the instrument 110 with the first metatarsal 202. In some aspects, a physician may manipulate the slider 154 and actuator 156 toward a bottom portion of the slot 152 prior to placing the aforementioned k-wire. Accordingly, after placement of the k-wire and coupling of the slider 154 with the first metatarsal 202, the slider 154 may be manipulated along the slot 152 in order address a rotational deformity of the first metatarsal 202 of the patient. Step 314 may also include placing a k-wire through at least one of the openings 168, 170 via at least one of the guides 172, 174 so as to couple the proximal portion 146 with the medial cuneiform 204. Prior to placement of the k-wire in the cuneiform, a physician may determine a desired fixation means (e.g., plate or intramedullary nail) and subsequently place the k-wire through the opening 168, 170 corresponding to the determined means of fixation. It should be understood that step 314 may also include a physician aligning the axes of the coupling members 164 and/or the coupling member 176 in a parallel configuration to a desired position of the long axis of the first metatarsal 202 so as to position the medial portion 142 to apply compression across the first tarsometatarsal joint in a direction parallel to the long axis.

Process 300 is shown to include a step 316 of manipulating the actuation instrument to reposition the first metatarsal from a first position to a second position, according to an exemplary embodiment. Step 316 may include a physician manipulating the slider 154 along the arcuate path of the slot 152 so as to rotate the first metatarsal 202 and correct any rotational deformity. In some aspects, the slot 152 may include indications marked on a surface thereof (e.g., degrees, etc.) such that the physician may manipulate the slider according to the indications in order to achieve a known derotation of the first metatarsal 202. Step 316 may also include manipulation of the coupling member 160 so as to position the medial portion 142 of the instrument 110 closer to the lateral portion 112 of the instrument. Such manipulation of the coupling member 160 may reduce an intermetatarsal angle (e.g., an angle formed between longitudinal axes of the first and second metatarsals 202, 206) and accordingly retain the first metatarsal 202 in a desired, corrected position (with rotational and/or angular deformities corrected). Further, step 160 may include implementation of the compression mechanism 150 on order to apply a compressive force axially (e.g., parallel to and along a longitudinal axis of a desired position of the first metatarsal 202) between resected surfaces of the first metatarsal 202 and the medial cuneiform 204. In some aspects, the application of the compressive force may be done iteratively at multiple points throughout process 300.

Process 300 is shown to include a step 318 of manipulating at least one component of the actuation instrument from an unlocked position to a locked position so as to retain the first metatarsal in the second position, according to an exemplary embodiment. Further, once a physician has reached a desired position of the slider 154 within the slot 152 (and accordingly, a desired rotational position of the first metatarsal 202, and/or vice-versa), the physician may manipulate the actuator 156 so as to retain the slider 154 in the desired position within the slot 152. Further, step 318 may include manipulation of the coupling member 160 so as to retain the medial portion 142 of the instrument 110 in the desired position (which was established in the step 316) relative to the lateral portion 112 of the instrument 110.

Process 300 is shown to include a step 320 of applying fixation across the first tarsometatarsal joint, according to an exemplary embodiment. Step 320 may be performed prior to or after any/all instrument systems and components of have been decoupled from anatomy of the patient including but not limited to those shown and described herein. Step 320 may also include the incorporation of additional hardware (for example, that incorporated by reference previously herein) that is configured to facilitate the application of the fixation. The fixation may include intramedullary nails, bone plates, fasteners, or a combination of several components including but not limited to those mentioned herein. Further, step 320 may include the application of compression across the first tarsometatarsal joint so as to compress resected surfaces of the first metatarsal 202 and the medial cuneiform 204. Accordingly, step 320 may include manipulation of the compression mechanism 150 and the coupling member 176 thereof so as to achieve the desired compression. Step 320 may also include manipulation of the lock 162 in order to retain the medial portion 142 in the desired compressive position.

Process 300 is shown to include a step 322 of decoupling the actuation instrument and retention member from the anatomy of the patient, according to an exemplary embodiment. Step 322 may include the manipulation of the lock 162 and/or one or more of the actuator 178 and/or the release 127 either prior or subsequent to removal of k-wires placed in the first metatarsal 202 and the medial cuneiform 204. Step 322 may include manipulation of one or more of the coupling members 126, 160, 164, and/or 176 in order to remove and/or disassemble portions of the instrument 110. Additionally, step 322 may include removal of the coupling element 140 from the shaft 138 of the retention member 130 such that the upper portion 116 may be decoupled from the retention member 130. The retention member 130 may be subsequently decoupled from the second metatarsal 206 either prior or subsequent to removal of the k-wire facilitating the coupling.

Process is shown to include a step 324 of closing the incision, according to an exemplary embodiment. Step 324 may include implementing one or more components common to closing surgical incisions, including adhesives and/or stitches/sutures/tapes. Further, this step may be performed before or after all hardware (e.g., instrumentation, systems, etc.) is removed from the patient, as this step may be performed iteratively and, as such, instrument/system/component removal may also occur iteratively.

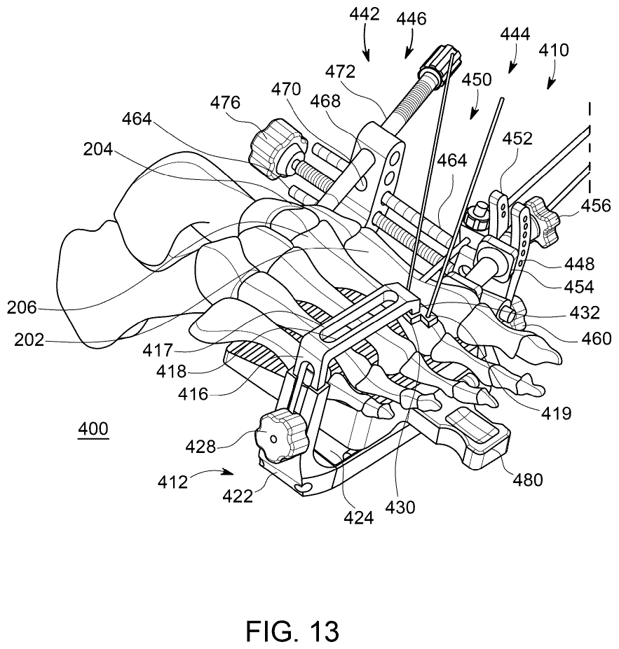

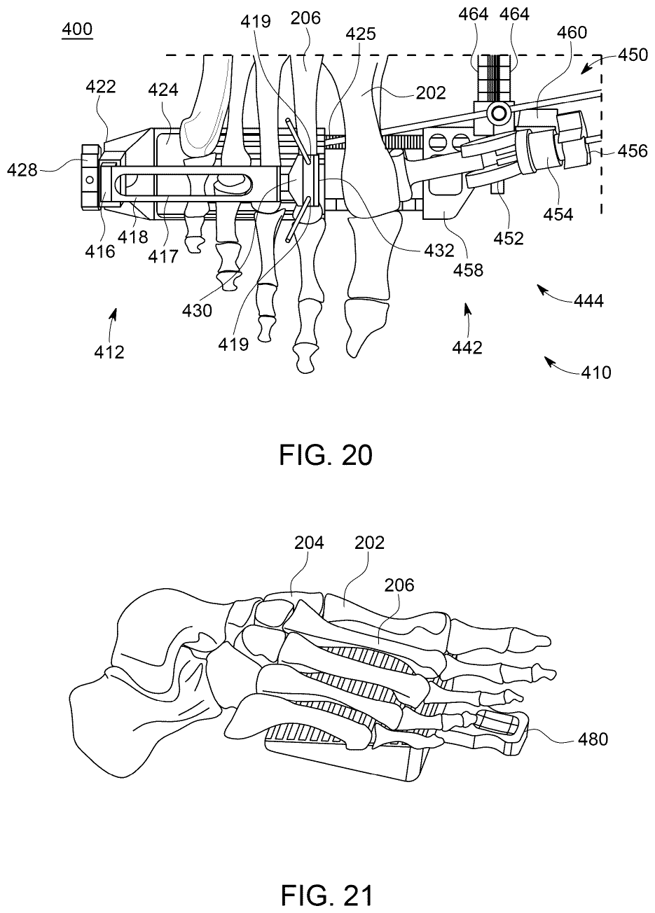

Referring now to FIGS. 12-20, an alignment instrument system 400 (referred to hereinafter as “instrument system 400” or “system 400”) is shown, according to an exemplary embodiment. The system 400 may be implemented to aid a physician in positioning, repositioning, or referencing various anatomy of a patient intraoperatively, particularly bony structures of the foot including, for example, a first metatarsal 202, a medial cuneiform 204, and a second metatarsal 206 of a patient. It should be understood, however, that implementation of the system 400 may include the system interfacing, releasably coupling, referencing, manipulating, or otherwise incorporating various structures of the foot or ankle of a patient including but not limited to the first metatarsal 202, the medial cuneiform 204, and the second metatarsal 206. Additionally, the system 400 may be implemented in conjunction with one or more systems (e.g., instrument systems, implant systems, etc.) and/or components thereof (e.g., implants, instruments, etc.) including but not limited to those incorporated by reference herein. Further, the system 400 may also be implemented in conjunction with various surgical methodology, for example for bunion procedures or other procedures to address anatomy of the midfoot and forefoot. As such, the system 400 may be implemented in conjunction with the surgical method shown and described subsequently herein, as well as other methodology.

The system 400 is shown to include an alignment instrument 410 (referred to hereinafter as “instrument 410”) and may also include the retention member 130, with the retention member 130 releasably couplable with the instrument 410 and/or other components of the system. Further, the retention member 130 is configured the same as or similar to that shown and described previously herein. The system 400 may also include various stabilization wires, also referred to as “k-wires” configured to facilitate coupling between one or more components of the system 400 (for example, the instrument 410 and the retention member 430) and bony anatomy of a patient (for example, the first metatarsal 202, the medial cuneiform 204, and the second metatarsal 206). As shown, the instrument 410 may have a substantially clamp-like geometry (e.g., opposing elements configured to be manipulated in order to apply a biasing force therebetween) and accordingly, clamp-like functionality (e.g., configured to be actuated in order to reposition or retain in a position one or more components with which the instrument interfaces). The instrument 410 may include one or more features configured to directly couple with the anatomy (e.g., interface with, contact, or otherwise address the anatomy) and/or may include one or more features configured to indirectly couple with the anatomy (e.g., by coupling with one or more intermediate components, which in turn couple with the anatomy).

The instrument 410 is shown to include a lateral portion 412 and a medial portion 442, where the lateral and medial portions 412, 442 are positioned substantially opposite the instrument 410 from one another. As shown in FIGS. 12-20, the lateral and medial portions 412, 442 may be releasably and translatably coupled with one another such that one or both components may be adjusted (e.g., translated in a single plane or along an axis) relative to the other. Further, the instrument 410 may be assembled and disassembled via the releasable coupling of the lateral and medial portions 412, 442.

The lateral portion 412 is shown to include a body 414 including an upper portion 416 substantially opposite the body 414 from a lower portion 422. The upper portion 416 may have a substantially L-shaped geometry (e.g., a geometry having one approximately 90-degree angle), and include an arm 417 extending medially (as shown in the configuration of FIGS. 1-10) from the angle. The arm 417 includes a cavity 418 therein and extending therethrough from a top surface of the arm 417 to a bottom surface and having a substantially oblong, elongated geometry. In some aspects, the arm 417 may include multiple cavities 418 disposed variously along the length of the arm 417. In the previous example, the multiple cavities 418 may be variously shaped and further may collectively have a similar footprint and lateral dimension to the cavity 418 as shown in FIGS. 12-20. The arm 417 also includes an extension 430 positioned at a terminal end of an integral with the arm 417. As shown in at least FIG. 12, the extension 430 may extend outward, downward, or in both directions from the arm 417 and may further include a platform 432 at an end of the extension 430 opposite that from the arm 417. The platform 432 may have a substantially rectangular geometry (but in some aspects, may also have other alternate geometries) with at least one opening 419 (shown in FIGS. 12-20 as a pair of openings and referred to as such hereinafter) extending therethrough. The openings 419 have a substantially circular/cylindrical geometry and extend through the platform 432 from a top surface to a bottom surface thereof (similar to the cavity). The openings 419 may be converging, diverging, or parallel with respect to central axes thereof and further may be configured to facilitate coupling between the platform 432 (and thus the arm 417 and instrument 410) with the anatomy of a patient, for example the second metatarsal 206. The upper portion 416 further includes a plurality of openings 420 disposed opposite the approximately 90-degree angle from the arm 417. As shown in at least FIG. 12, the openings 420 may be arranged vertically and have equal dimensions and vertical spacing therebetween. Further, the openings 420 may also include a larger opening than those having equal size and vertical spacing, with the larger opening positioned below the equally sized openings and configured to receive at least a portion of a fastener 428 therein and/or therethrough so as to facilitate coupling of the upper and lower portions 416, 422 of the body 414. The larger opening of the openings 420 may also include a threading on an inner portion thereof (e.g., to interface with a complementary threading of the fastener 428) and be configured to, in conjunction with the fastener 428, retain the upper portion 416 at a desired height as the upper portion 416 extends from a portion of the lower portion 422. Accordingly, actuation of the fastener 428 may permit vertical translation of the upper portion 416 relative to the lower portion 422 and/or retain the upper portion 416 in a desired position (e.g., at a desired height) relative to the lower portion 422. Such actuation of the fastener 428 and/or the upper and lower portions 416, 422 may be performed in order to adjust a height of the lateral portion 412 according to a size and height of a forefoot and/or midfoot of a patient to achieve a position the same or similar to that as shown in at least FIGS. 12-18.

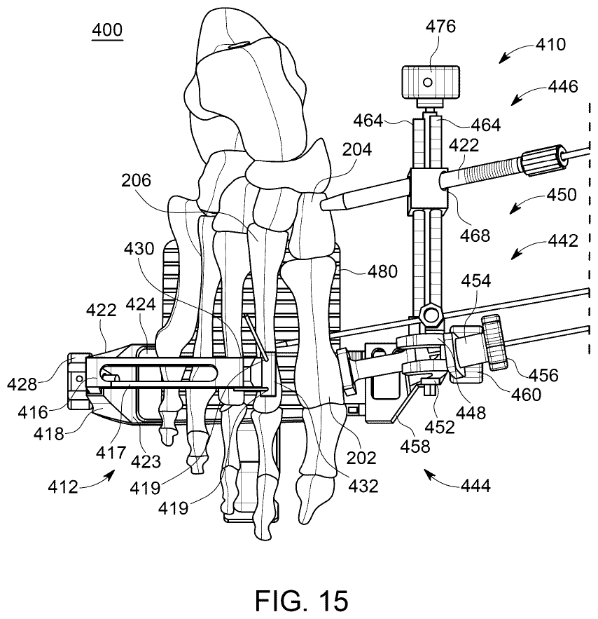

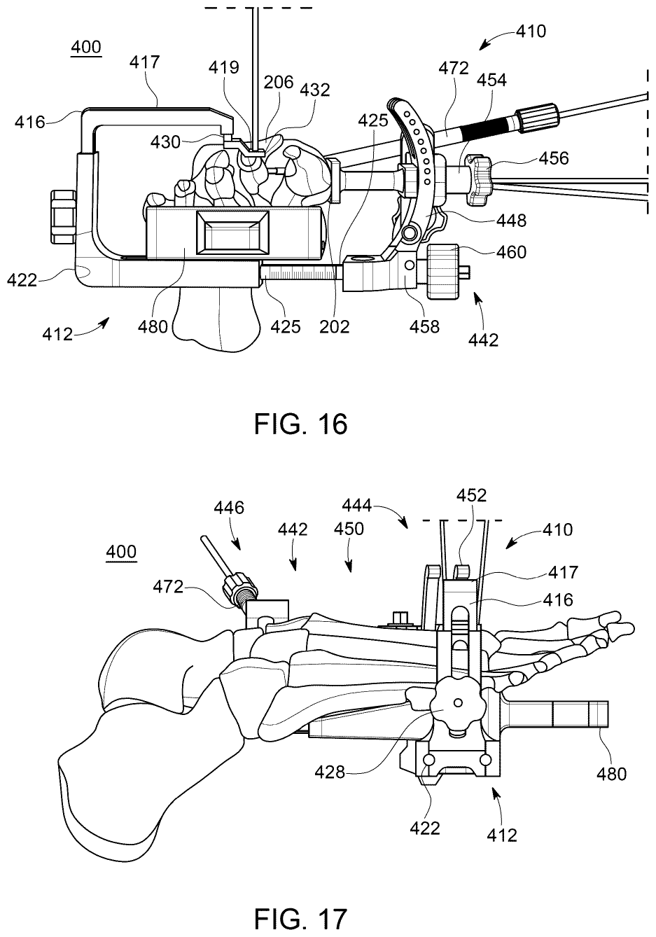

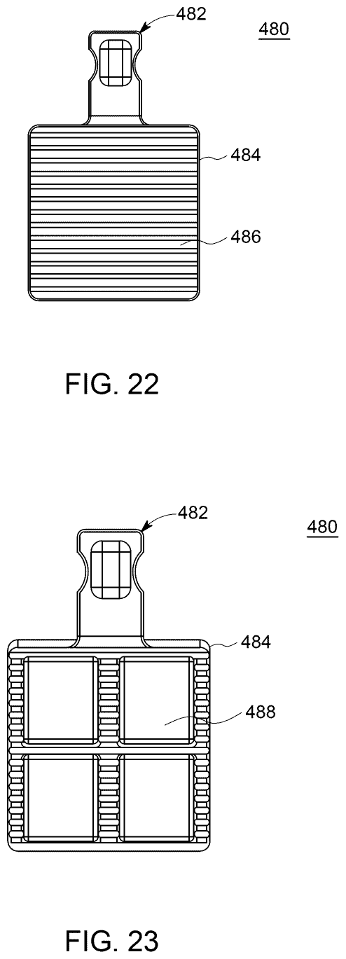

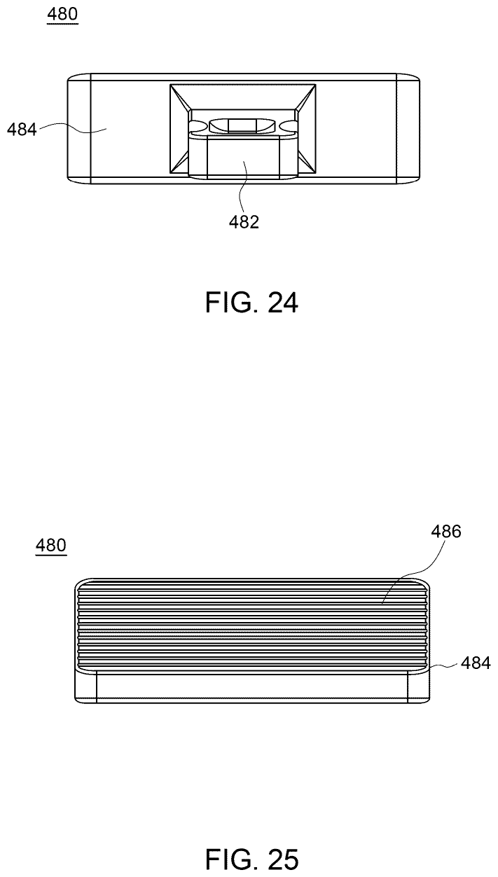

The lower portion 422 of the body 414 is shown to have a similar geometry to that of the upper portion 416 in that the lower portion 422 has a geometry that includes an approximate right angle, according to the exemplary embodiment of FIGS. 12-20. The lower portion 422 as shown is configured to receive at least a portion of the upper portion 416 therein so as to facilitate the aforementioned adjustment and translation therebetween. The lower portion 422 includes an at least partially open feature on a lateral-most portion thereof so as to facilitate placement of the fastener 428 within the largest of the openings 420, as well as any desired placement of a k-wire through one or more of the remaining openings 420. Opposite the approximately 90-degree angle from the at least partially open feature, the lower portion 422 includes an arm 423 extending in a plane that, when coupled with the upper portion 416, is substantially parallel to the plane in which the arm 417 extends. Further, the arm 423 extends laterally and as shown in FIG. 15, the footprint of the arm 417 is substantially centered within the footprint of the arm 423. The arm 423 is also shown to include a cavity 424 disposed therein and extending at least partially along a length thereof. Similar to the cavity 418, in some aspects the cavity 424 may include multiple cavities 424 which may occupy a substantially similar length of the arm 423 to the single cavity 424 as shown at least FIGS. 15-16. In some aspects, the arm 423 may include a texture disposed on at least a portion of an upper surface thereof, shown in FIG. 19 as a plurality of elongated, raised features. Such texture may be configured to be complimentary to or otherwise engageable with a complimentary texture disposed on a bottom portion of an instrument 480, shown and described with reference to at least FIGS. 21-27.

The lower portion 422 is further shown to include an opening 425 and a coupling member 426 disposed at a terminal end of the arm 423. The coupling member 426, as shown, may be threadably coupled in an opening the same as or similar to the opening 425. Further, the coupling member 426 may be configured to releasably and threadably couple with a complementary coupling feature of the medial portion 442 so as to facilitate the aforementioned releasably coupling and translatability between the lateral and medial portions 412, 442. The opening 425 may be configured to receive (at least partially therethrough/therein) and releasably and threadably couple with a component the same as or similar to the coupling member 426, for example a coupling member of the medial portion 442. Similarly, the coupling member 426 and a complementary component of the medial portion may, collectively, facilitate both releasably coupling and adjustable translation between the lateral and medial portion 412, 442. For example, the coupling member 426, the opening 425, and complementary components of the medial portion 442 may facilitate adjustment of the instrument 410 in the medial-lateral direction such that the instrument 410 may have an adjustable width to accommodate various sizes/widths of feet of the patient. In some aspects, the lower portion 422 may include an actuator (e.g., a button, switch mechanism, etc.) configured to facilitate the releasable coupling of the lateral and medial portions 412, 442 with respect to the opening 425, the coupling member 426, and other complementary components of the medial portion 442. For example, the actuator may be manipulatable from a first, locking/retaining position in which decoupling or translation of the lateral and/or medial portions 412, 442 is not permitted to a second, unlocked position in which decoupling or translating the lateral and medial portions 412, 442 is permitted (e.g., the aforementioned adjustment may occur).

The medial portion 442 includes a distal portion 444 substantially opposite the medial portion 442 from a proximal portion 446, according to an exemplary embodiment. The distal portion 444 may be releasably and translatably coupled with the proximal portion 446 via one or more coupling members and/or mechanisms, which may be the same as or similar to those facilitating the releasable and translatable coupling between the lateral and medial portions 412, 442.

The distal portion 444 includes an upper portion 448 and a lower portion 458. As shown, the upper and lower portions 448, 458 are integral with one another but in some aspects may be releasably couplable with one another. The upper portion 448 may have a substantially curved geometry as it extends upward from the lower portion 458, where the curvature extends outward medially before curving laterally (e.g., a convex geometry facing medially and a concave geometry facing laterally). Further, the upper portion 448 may be positioned as a substantially oblique angle relative to a longitudinal axis of the medial portion 442 that extends substantially in the anterior-posterior direction. The upper portion 448 is shown to include a slot 452 extending vertically along an upper portion thereof and, as shown, is open on its upper-most edge so as to facilitate insertion and/or removal of a component into the slot 452. As shown, the slot 452 defines a plane (or series of planes) extending therethrough that forms a substantially oblique angle with the coupling member 426 and plane or longitudinal axis thereof.

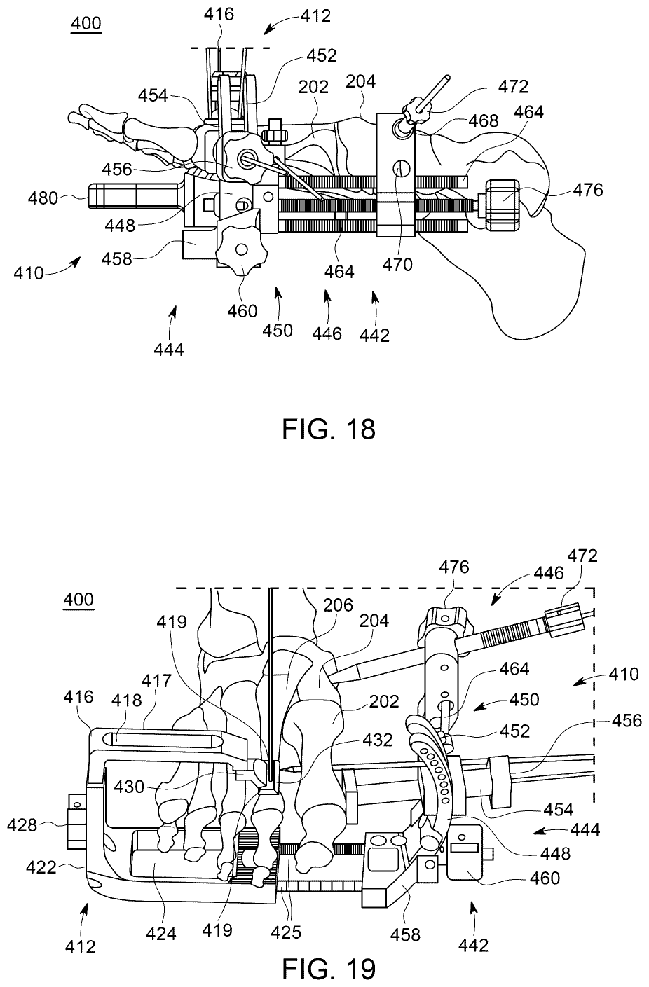

The upper portion 448 includes a slider 454 positioned at least partially within the slot 452 and having a geometry along at least a portion of a length thereof having a lateral dimension less than the width of the slot 452. Accordingly, the slider 454 may be positioned at least partially within the slot 452 and translated in a substantially vertical direction along an arcuate path defined by the aforementioned geometry of the upper portion 448. As shown, the slider 454 may include a variety of features and/or components along a length thereof, which may also include various lateral dimensions. For example, on opposite sides of the portion of the slider 454 with a lateral geometry lesser than that of the slot 452, the slider 454 may include geometric features having a lateral dimension greater than that of the slot, so as to retain the slider 454 within the slot 452 (from a medial-lateral perspective). Further, the slider 454 may be configured to be cannulated so as to receive a component therein and at least partially therethrough (for example, a k-wire) the cannulation, which may extend along a longitudinal axis of the slider 454. The slider 454 may also include one or more surfaces, for example at a terminal end, with a contoured geometry and/or surface configured to facilitate interfacing with the bony anatomy of a patient (for example, the first metatarsal 202). The slider 454 is also shown to include an actuator 456 disposed on a medial-most portion of the slider 454 (as shown in FIG. 18). The actuator 456 may also include a cannulation configured to align with the cannulation of the slider 454 such that a single k-wire may be received into and through both components (for example, to releasably couple the slider 454 and actuator 456 with the first metatarsal 202 and facilitate manipulation of the first metatarsal 202 via actuation of the slider 454 along the path defined by the slot 452). The actuator 456 may be manipulatable from a first position in which the slider 454 is translatable along the arcuate path (which, in some embodiments, may be a substantially vertical path) defined by the slot 452 of the upper portion 448 to a second position in which the slider 454 is retained in a desired position along the arcuate path of the slot 452.

The lower portion 458 is shown to include a coupling member 460 which is shown to extend through an opening in the lower portion 458 from the medial side to the lateral side, as shown in at least FIG. 19. In some aspects, the coupling member 460 may include one or more features the same as and/or similar to those of the coupling member 426 as shown and described previously. As shown, the coupling member 460 is positioned posterior relative to the coupling member 426 and, further, is configured in a substantially parallel orientation to the coupling member 426. The coupling member 460 may include an actuator (shown as a knob) which is positioned on the medial side of the lower portion 458 while an elongated portion of the coupling member 460 (which may include a threading) extends into and through the lower portion 458 to be received at least partially within the opening 425 of the lateral portion 412 so as to facilitate coupling between the lateral and medial portions 412, 442. Rotation of the knob of the coupling member 460 may facilitate engagement/disengagement of the coupling member 460 with threading of openings in the lower portion 458 and the lateral portion 412. Similarly, the lower portion 458 may include an opening the same as and/or similar to the opening 425 that is configured to receive at least a portion of the coupling member 426 therein. Collectively, the aforementioned openings and coupling members 426, 460 are configured to facilitate coupling between the lateral and medial portions 412, 442 as well as translatable adjustment in the medial-lateral direction to accommodate a size and/or width of a foot of the patient. In some embodiments, the coupling members 426, 460 may both be coupled with either the lateral portion 412 or the medial portion 442 prior to coupling with the complementary component. Further, one or more of the coupling members 426, 460 may be removable from the lateral and medial components 412, 442 entirely so as to facilitate disassembly of the instrument 410.

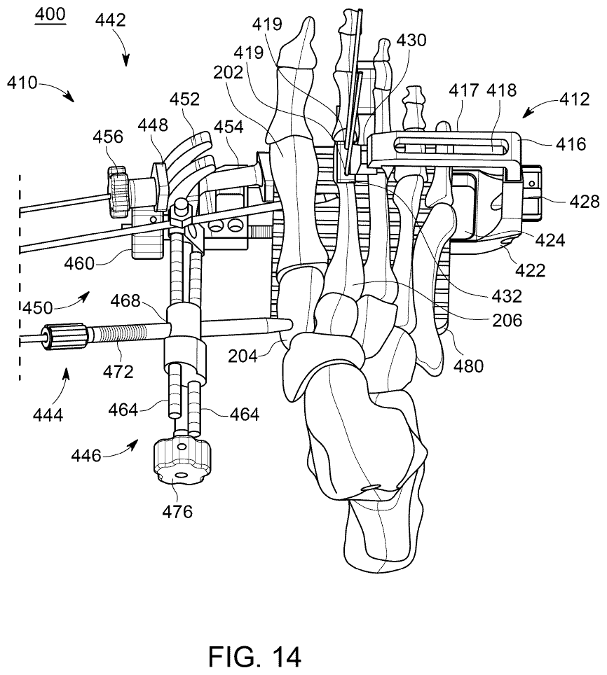

As shown in at least FIG. 14, the proximal portion 446 has a similarly curved geometry to that of the upper portion 448 of the distal portion 444 (although the proximal portion 446 may include an alternate or more gradual arcuate shape). The proximal portion 446, which is shown to be coupled with the distal portion 444, is shown to include an upper opening 468 and a lower opening 470 extending through the proximal portion 446 from a medial surface to a lateral surface. As shown, the openings 468, 470 include a substantially circular and cylindrical geometry so as to receive at least a portion of at least one of an upper guide 472 and/or a lower guide (not shown, but the same as or similar to the lower guide 174) therein and therethrough. The guide 472 may be interchangeable in that the guide 472 may have compatibility with each of the openings 468, 470. Further, the guide 472 may include a cannulation along a longitudinal axis thereof configured to receive a coupling element, for example a k-wire, therein and therethrough (for example, to facilitate coupling of the proximal portion 446 with the medial cuneiform 204). As shown in FIG. 18, the upper opening 468 includes an indication of the word “nail” adjacent to the opening 468 on a medial surface of the proximal portion 446, whereas the lower opening 470 includes an indication of the word “plate” adjacent to the opening 470 on a medial surface of the proximal portion 446. The aforementioned indications may be configured to guide optimal placement of a k-wire through one of the openings 468, 470 (via guide 472) and into the medial cuneiform 204 so as to couple the proximal portion 446 with the medial cuneiform 204 and also avoid any space needed (e.g., surfaces of anatomy or areas above the foot of the patient) to implement any systems for applying fixation across the first tarsometatarsal joint (for example, a plate or nail system, where a physician would place the aforementioned k-wire in the opening 468, 470 corresponding to the desired fixation means).

The distal portion 444 is slidably (e.g., translatably) and threadably coupled with the proximal portion 446 and, as shown in at least FIG. 18, are coupled via a compression mechanism 450 disposed therebetween. The compression mechanism 450 is shown to include a pair of coupling elements 464, shown in FIG. 18 as elongated threaded members, configured to threadably engage and couple with the distal and proximal portions 444, 446 via openings on side surfaces thereof which may be the same as and/or similar to the opening 425 as shown and described previously. As shown, the coupling elements 446 are positioned substantially parallel to one another and in a co-planar configuration. In some aspects, the coupling elements 464 may extend into and through the proximal portion 446 as shown in FIG. 18. The coupling elements 464 may be configured to facilitate adjustment of the medial portion 442 and the distal and proximal portions 444, 446 thereof such that a physician may adjust the medial portion 442 of the instrument 410 according to a size and/or length of the foot of the patient. Further, the compression mechanism 450 includes a coupling member 476 positioned vertically between the coupling elements 464 and engaging with the distal and proximal portions 444, 446 via the same or similar openings disposed on side surfaces of the distal and proximal portions 444, 446. The coupling member 476 may be the same as or similar to the coupling member 460 and include a knob portion as well as an elongated portion extending from the knob and having a threading along at least a portion thereof. Actuation of the knob of the coupling member 476 may be configured to engage the threading thereof with complementary threading of at least one of the distal and proximal portions 444, 446. For example, if the distal portion 444 is coupled with the first metatarsal 202 of a patient and the proximal portion 446 is coupled with the medial cuneiform 204 of the patient, rotation of the knob of the coupling member 476 is configured to apply a compressive (or, if rotated in the opposite direction, a distractive) force across the first tarsometatarsal joint so as to bias the first metatarsal 202 and the medial cuneiform 204 (which may include resected surfaces thereof) toward or away from one another. The distal portion 444 is shown to include a locking button 462, which may be configured to retain the medial portion 442 in the aforementioned compressive state when engaged by a physician (e.g., so fixation can be applied with the first metatarsal 202 and medial cuneiform 204 are being biased toward one another). In some aspects, the proximal portion 446 may include an actuator (e.g., a button or switch mechanism) which, when actuated, may enable disassembly of the medial portion 442 and components thereof.

Referring now to FIGS. 11-18 and 21-27, an instrument 480 is shown. The instrument 480 may be implemented in conjunction with (and engage with) the instruments 110, 410, as well as other surgical instruments. The instrument 480 is shown to include a handle 482 extending from a body 484 at a substantially orthogonal angle to a surface of the body 484, where the handle 482 is ergonomically shaped to be grasped by a user. For example, the handle 482 may include one or more narrowed or widened sections along the length thereof configured to facilitate gripping by a user. Further, the handle 482 may also include one or more textures or protrusions on one or more surfaces thereof, for example the top and bottom, configured to assist a user in gripping and manipulating the instrument 480.

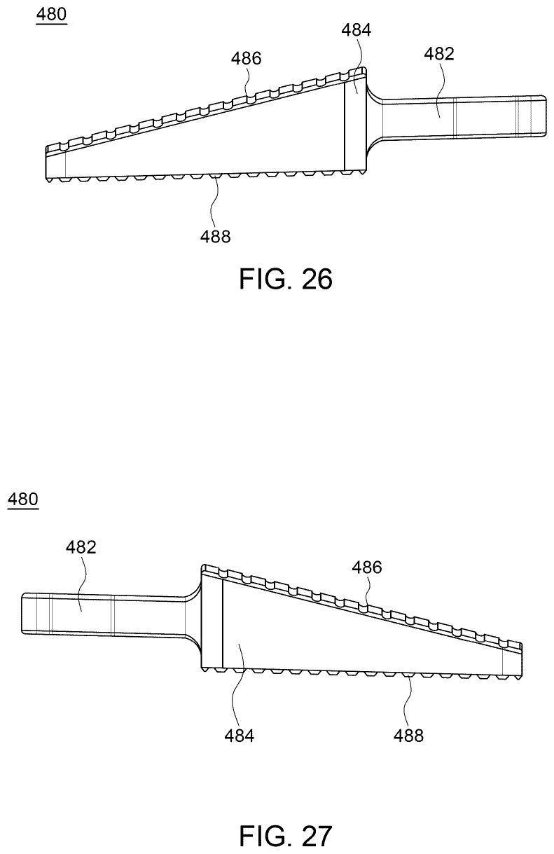

The body 484 of the instrument is shown to be substantially wedge-shaped, and, when viewed from a side perspective as shown in FIGS. 26-27, includes at least one substantially right angle. The body 484 is shown to include a top and bottom surface 486, 488 opposite the body 484 from one another and positioned in planes forming an oblique angle to one another. The top surface 486 is shown to include a texture disposed on at least a portion thereof, which may be configured to facilitate engagement with the bottom of the foot of the patient, and thus retain the instrument 480 in the desired position intraoperatively (as shown in FIG. 12, for example). The texture on the top surface 486 as shown includes a plurality of raised members extending from one side of the body 484 to the other (which may be horizontal, vertical, or at various oblique angles to the edges of the top surface 486). In some aspects, this texture may include depressions or alternating raised and depressed portions of the top surface 486. Further, various patterns may also be implemented as a texture on the top surface 486, for example protrusions arranged in one or both of a randomized configuration or a pattern layout (e.g., repeating configurations).

The bottom surface 488 is also shown to include a texture disposed thereon, as shown in at least FIG. 23. The texture disposed on the bottom surface 488 may include raised members the same as and/or similar to those shown and described with reference to the top surface 486, and may also include additional components. For example, the bottom surface 488 may include one or more ground contacts, shown as rectangular protrusions in FIG. 23, configured to optimize stability of the instrument 480 when receiving a force from the foot of the patient. Further, the instrument 480 may be positioned by the physician between the bottom of the patient's foot and the instrument 410 as shown in at least FIG. 16 and, accordingly, the bottom surface 488 (and textured elements thereof) may be configured to have a complimentary geometry to a texture disposed on the top surface of the arm 423 (and/or other adjacent components). For example, the arm 423 may include alternating raised members, depressions, or a combination of the two configured to engage with the same or a similar texture on the bottom surface 488 so as to ensure retention of the instrument in a desired position relative to the foot of the patient and the instrument 410.

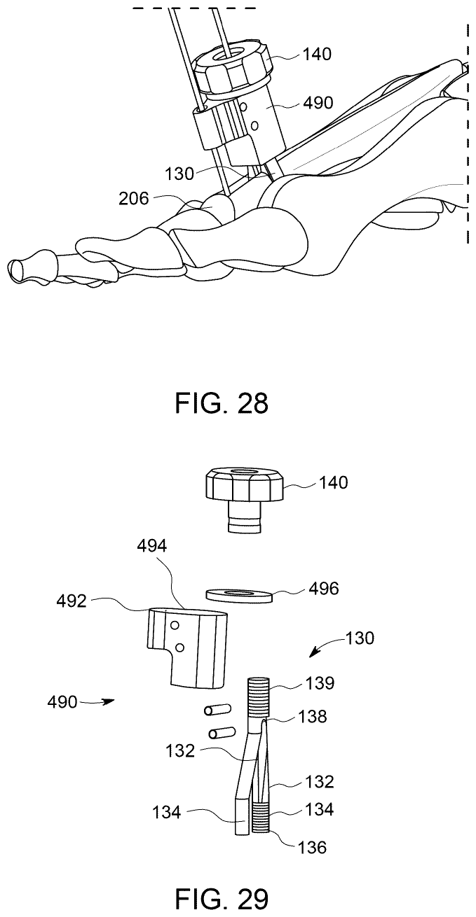

Referring now to FIGS. 28 and 29, the retention element 130 is shown adjacent to components with which it may be implemented in conjunction with the system 400 and the instrument 410. The retention element 130 may be couplable with a guide block 490, where the guide block 490 includes a first opening 492 spaced from a second opening 494 with the second opening 494 extending from a top surface through to a bottom surface of the guide block 490 and configured to receive at least a portion of the retention element (e.g., the shaft 138) at least partially therein. The opening 492 may be configured to receive a k-wire therethrough so as to facilitate coupling of the guide block 490 with a bone (e.g., the second metatarsal 206) of a patient. The retention element 130 may be releasably coupled with the guide block 490 when the shaft 138 is received into and through the opening 494, with the upper portion of the shaft 138 and at least a portion of the threading 139 protruding from the upper portion of the opening 494. Accordingly, a washer 496 may be placed over the shaft 138 such that it abuts the top surface of the guide block 494, and the coupling element 140 may be threadably coupled with the threading 139 of the retention element 130.

In some aspects, the guide block 490 may be implemented in conjunction with the instrument 110 and/or 410 in order to facilitate the placement of one or more k-wires or otherwise facilitate coupling of the instruments 110, 410 with anatomy of the patient. For example, k-wires may be placed in the first opening 492 of the guide block and through the cannulation of the retention element 130 (and thus, through the second opening 494 of the guide block) to couple the components with the second metatarsal 206 of the patient. The retention element 130 and guide block 490 may then be decoupled from the patient with the k-wires remaining coupled with the second metatarsal 206. The instrument 110 and/or 410 may then be releasably coupled with the second metatarsal 206 via the already placed k-wires, for example via the extension 430 and platform 432, with the k-wires received into and through the openings 419.

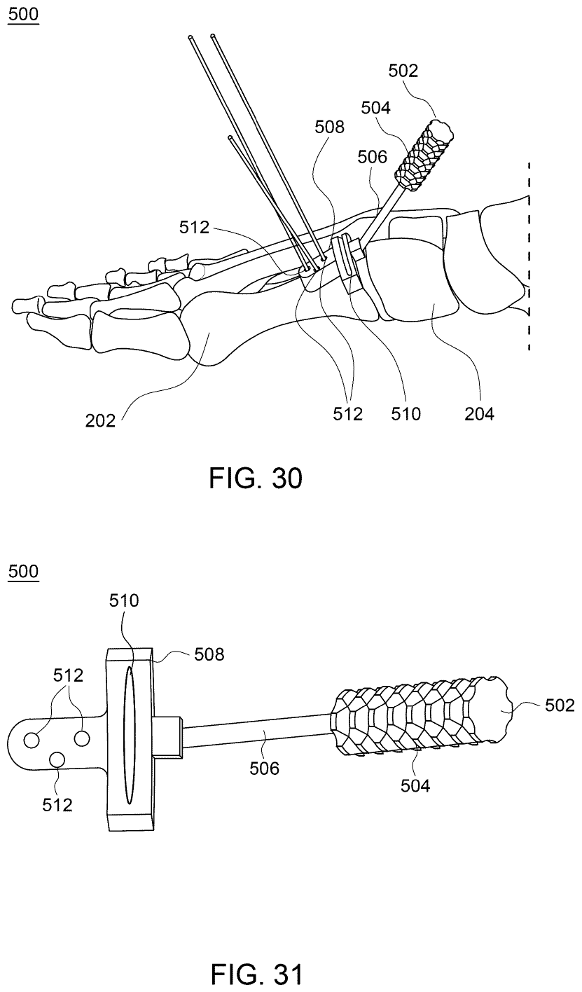

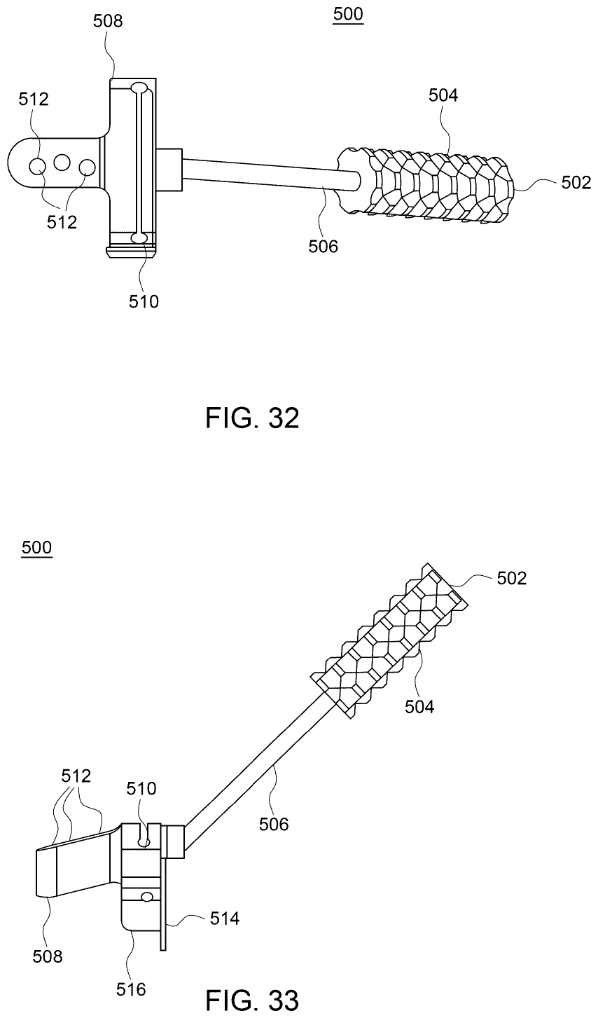

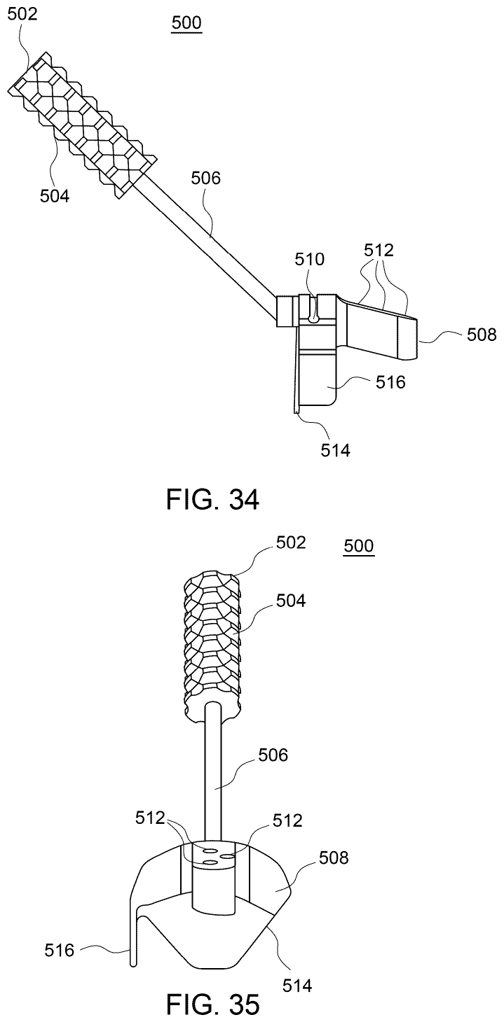

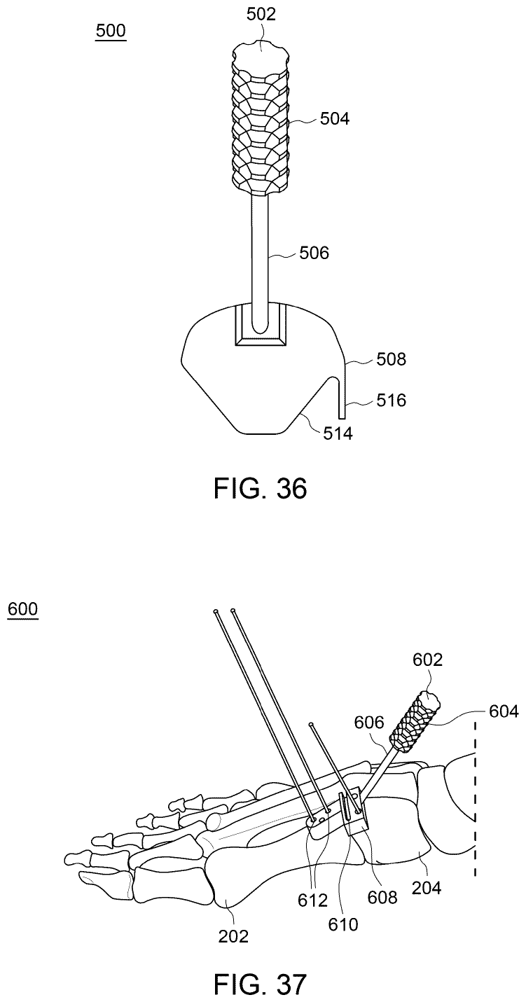

Referring now to FIGS. 30-36, a resection instrument 500 which may be implemented in conjunction with the systems 100, 400, is shown. The instrument 500 is shown to include a handle 502 separated from a body 508 by a shaft 506. The handle 502 is shown to include a texture 504 disposed on at least a portion thereof. The shaft 506 is shown to be integral with both the handle 502 and the body 508. The body 508 is shown to include at least one cut slot 510 disposed in a central portion thereof and extending substantially from a first side of the body 508 to an opposite side thereof. The body 508 further includes a protrusion extending from the body 508 on a side substantially opposite from the shaft 506, with the protrusion including at least one opening 512 (shown as three openings in FIG. 32) extending from a top surface therethrough to a bottom surface. In some aspects, two or more of the openings 512 may have central axes parallel to one another (which may be perpendicular to the top surface of the body 508). Further, in some aspects the openings 512 may have central axes arranged at oblique angles relative to one another and/or the top surface of the body 508. The body 508 further includes a paddle 514 protruding in a downward direction from the bottom surface of the body 508 in a plane substantially parallel to the at least one cut slot 510. The paddle 514 is integral with a cut protector 516 which extends from the bottom portion of the body 508 and is positioned at a perpendicular or oblique angle relative to the cut slot 510 and/or the paddle 514. In some aspects, the resection instrument 500 may be couplable with the first metatarsal 202 of the patient via k-wires inserted through the one or more openings 512. A cutting tool may then be manipulated within the cut slot 510 so as to resect a portion of the first metatarsal 202, with the cut protector 516 configured to limit a depth of the cutting tool and protect the anatomy adjacent to the desired cutting area.

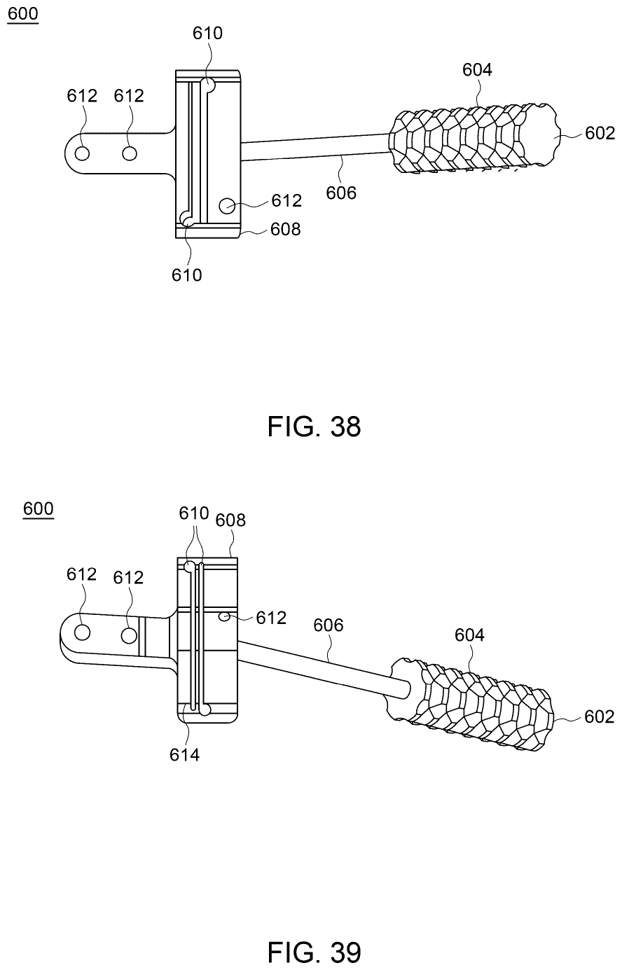

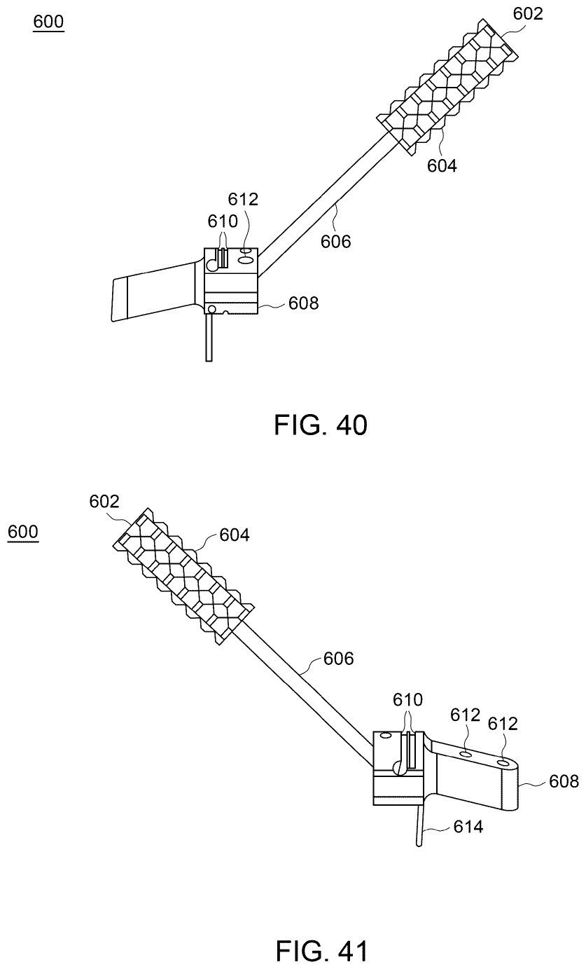

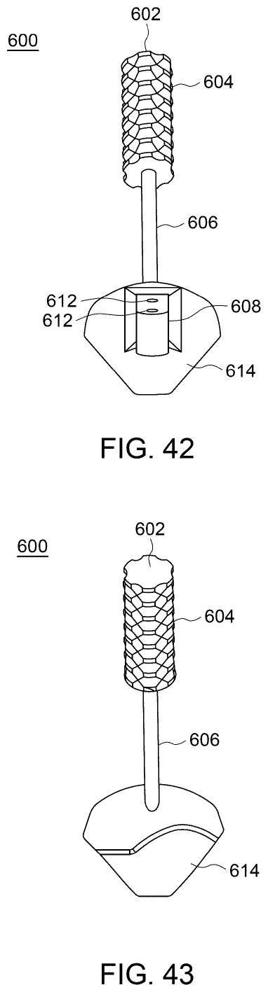

Referring now to FIGS. 37-43, a resection instrument 600 which may be implemented in conjunction with the systems 100, 400, is shown. The instrument 600 is shown to include a handle 602 separated from a body 608 by a shaft 606. The handle 602 is shown to include a texture 604 disposed on at least a portion thereof. The shaft 606 is shown to be integral with both the handle 602 and the body 608. The body 608 is shown to include at least one cut slot 610 (shown as two cut slots) disposed in a central portion thereof and extending substantially from a first side of the body 608 to an opposite side thereof. The body 608 further includes a protrusion extending from the body 608 on a side substantially opposite from the shaft 606, with the protrusion including at least one opening 612 (shown as two openings in FIG. 39) extending from a top surface therethrough to a bottom surface, as well as an opening 612 positioned between the cut slots 610 and the shaft 606. In some aspects, two or more of the openings 612 may have central axes parallel to one another (which may be perpendicular to the top surface of the body 608). Further, in some aspects the openings 612 may have central axes arranged at oblique angles relative to one another and/or the top surface of the body 608. The body 608 further includes a paddle 614 protruding in a downward direction from the bottom surface of the body 608 in a plane substantially parallel to the at least one cut slot 610. In some aspects, the resection instrument 600 may be couplable with the first metatarsal 202 of the patient via k-wires inserted through the one or more openings 612. A cutting tool may then be manipulated within the cut slot 610 so as to resect a portion of the first metatarsal 202. In some aspects, the instrument 600 may be couplable with a first metatarsal 202 and/or a medial cuneiform 204 of a patient via k-wires inserted through the one or more openings 612. A cutting tool may then be manipulated within the cut slot 610 so as to resect a portion of the medial cuneiform 204.

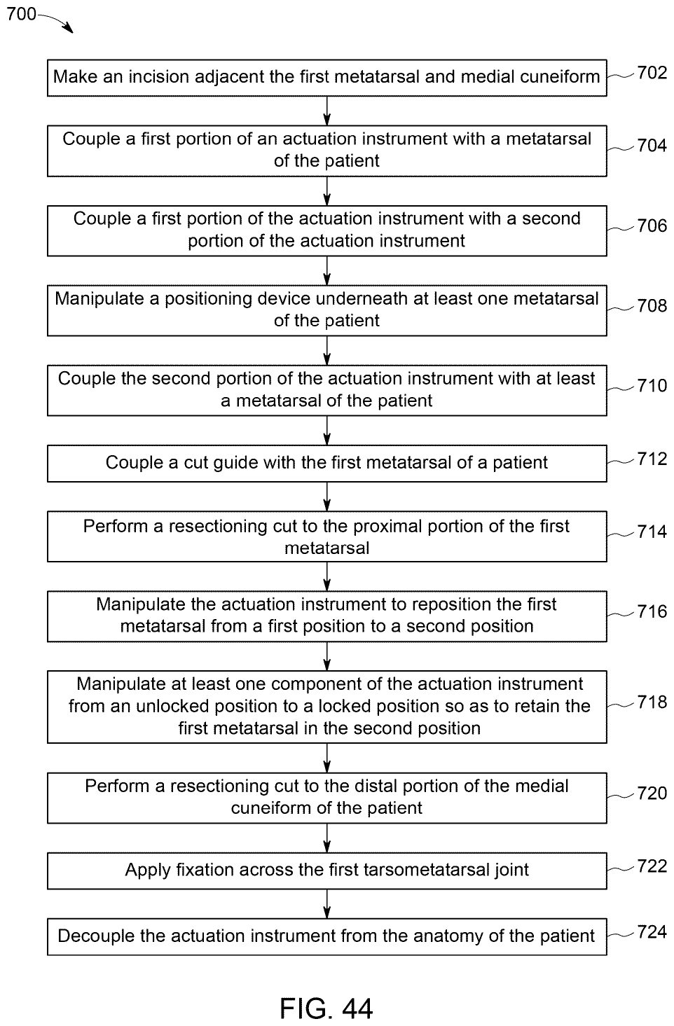

Referring now to FIG. 44, a process 700 for performing at least a portion of a bunion procedure is shown, according to an exemplary embodiment. In performing process 700, the systems 100, 400, and/or one or more components thereof (in addition to other systems/components) may be implemented. Further, it should be understood that in performing process 700, one or more of the steps thereof may be omitted, repeated, performed in an alternate sequence, or replaced with one or more alternate steps.

Process 700 is shown to include a step 702 of making an incision adjacent to the first metatarsal 202 and the medial cuneiform 204, according to an exemplary embodiment. In some aspects, one or more components of the system 100 and/or 400 may be adjacent to anatomy of the patient when the physician performs step 702. Further, the incision made in step 702 may be positioned so as to facilitate placement and coupling of a specific cut guide (including, for example, those shown and described with reference to FIGS. 30-43 as well as those incorporated by reference herein) with/adjacent to the first tarsometatarsal joint (e.g., to couple a first portion of the guide with the first metatarsal 202 and a second portion of the guide with the medial cuneiform 204).

Process 700 is shown to include a step 704 of coupling a first portion of an actuation instrument with a metatarsal of the patient, according to an exemplary embodiment. In some aspects, the actuation instrument (e.g., the instruments 110 and/or 410, which may be referred to with reference to the process 700 as actuation instruments 110 and/or 410) may be coupled with the metatarsal of the patient (e.g., the second metatarsal 206) using the retention member 130 as well as other associated components. In other aspects, the actuation instrument may be coupled with the metatarsal of the patient (e.g., the second metatarsal 206) as shown in at least FIG. 12, with one or more k-wires placed through openings 419 of the platform 432 (which may be along converging, diverging, or otherwise oblique axes/trajectories) and into the second metatarsal 206 to achieve the coupling.

Process 700 is shown to include a step 706 of coupling the first portion of the actuation instrument with a second portion of the actuation instrument, according to an exemplary embodiment. The step 706 may include, for example, coupling the lateral portion 412 of the instrument 410 with the medial portion 442 via the opening 425 and the coupling member 426, and may also include other coupling aspects in some embodiments. Further, in coupling the lateral and medial portions 412, 442, a user (e.g., a physician, surgical technician. etc.) may adjust the size of the actuation instrument to adjust the width and/or height (e.g., in a medial lateral and/or a dorsal/plantar direction using the components/mechanisms described previously herein) to appropriately size the actuation instrument to the affected foot of the patient.

Process 700 is shown to include a step 708 of manipulating a positioning device underneath at least one metatarsal of the patient, according to an exemplary embodiment. The step 708 may include positioning, as shown in at least FIG. 14, an instrument such as the instrument 480 (e.g., by grasping the handle 482) between a portion of the actuation instrument 410 and the bottom of the foot of the patient. In some aspects, the step 708 may also include positioning the top surface 486 of the instrument 480 against a bottom surface of the foot of the patient such that the texture of the top surface 486 contacts the bottom surface of the foot of the patient (and thus facilitates retention of the instrument 480 in the desired position relative to the foot of the patient). The step 708 may also include positioning the bottom surface 488 of the instrument 480 against a portion of the actuation instrument 410, such that the texture of the bottom surface 488 contacts a portion of the actuation instrument 410 (and any complimentary texture on portions thereof, for example the top surface of the arm 423), and thus retains the instrument 480 in a desired position relative to the actuation instrument 410.

Process 700 is shown to include a step 710 of coupling the second portion of the actuation instrument with at least a metatarsal of the patient, according to an exemplary embodiment. The step 710 may include coupling the actuation instrument 410 with the first metatarsal 202 and/or the medial cuneiform 204 of the foot of the patient, for example as shown in at least FIG. 12, with one or more k-wires placed into the aforementioned bones though one or more of the cannulation of the slider 454, one or more openings disposed on the distal portion 444, and/or a guide (e.g., upper guide 472) within the upper and/or lower openings 468, 470.

Process 700 is shown to include a step 712 of coupling a cut guide with the first metatarsal 202 of a patient, according to an exemplary embodiment. Step 712 may include implementing a cut guide that is not couplable with other components of the system 100 and/or 400, for example one or more of the cut guides of FIGS. 30-43 such as the instrument 500. The cut guide may be coupled with the first metatarsal 202 (and in some aspects, also coupled with the medial cuneiform 204) via k-wires.

Process 700 is shown to include a step 714 of performing a resection cut to a proximal portion of the first metatarsal, according to an exemplary embodiment with the resection cut made with a sagittal or reciprocating saw so as to create a flat surface, for example a surface that is substantially parallel with the paddle 514 of the instrument 500. In some aspects, the cut plane of the cut made in the step 714 may be in a plane perpendicular to a longitudinal axis of the first metatarsal 202. In some alternate embodiments, the cut guide implemented in the step 712 may be configured to guide the cuts to the first metatarsal 202 to a desired angle or obliquity (e.g., configure the flat surfaces resulting from the cuts to have a known angle between one another). Following the resection cut of the step 714, the cut guide and any k-wires coupling the cut guide with the first metatarsal 202 may be decoupled and removed, along with any resultant bone separated from the first metatarsal 202 by the resection cut.

Process 700 is shown to include a step 716 of manipulating the actuation instrument to reposition the first metatarsal from a first position to a second position, according to an exemplary embodiment. The step 716 may include manipulating the first metatarsal 202 using one or more of the correction mechanisms of the actuation instrument 410, depending on the deformity of the patient and the correction desired by the physician. For example, the actuation instrument may be manipulated in the medial-lateral direction (e.g., such that the lateral and medial portions 412, 442 have the distance between one another decreased) to address an angular deformity of the first metatarsal 202. Further to the previous example, the physician may measure or approximate an angle between longitudinal axes of the first and second metatarsal 202, 206 to be 20 degrees, and may thus actuate the actuation instrument as mentioned previously using the coupling member 460 to decrease the measured angle to a desired (lesser) measurement or approximation. The physician may also, for example, manipulate the slider 454 within the slot 452 to address a rotational deformity of the first metatarsal 202. The two corrective steps shown and described previously may be performed iteratively, in various orders, or be repeated as deemed necessary by the physician to achieve a desired corrected position. It should be understood that, in some aspects, a physician may only desire to correct an angular or a rotational deformity and, accordingly, may not perform the aforementioned actions to address a deformity that the physician does not wish to correct.

Process 700 is shown to include a step 718 of manipulating at least one component of the actuation instrument from an unlocked position to a locked position so as to retain the first metatarsal in the second position, according to an exemplary embodiment. Following the manipulation of the first metatarsal 202 from a deformed position to a correction position in the step 716, the physician may manipulate the actuation instrument in order to retain the first metatarsal 202 in the corrected position. Accordingly, when a desired corrected rotational position has been achieved, the physician may actuate the actuator 456 so as to lock (e.g., retain) the slider 454 and, accordingly, the first metatarsal 202 in the corrected position. Similarly, once the desired angular corrected position has been achieved, the physician may enable one or more retention features (e.g., locking features) on the actuation instrument 410 to ensure that the affected anatomy (e.g., the first metatarsal 202) remains in the appropriate corrected position by “locking” the clamp in a position configured to retain the first metatarsal 202 in the corrected angular position.

Process 700 is shown to include a step 720 of performing a resection cut to the distal portion of the medial cuneiform of the patient, according to an exemplary embodiment. The step 720 may include, or be preceded by, a step of coupling at least a portion of an instrument, for example the cut guide 600, with at least the medial cuneiform 204 using one or more k-wires. In some aspects, the cut guide 600 may also be coupled with the adjacent first metatarsal or another adjacent bone so as to provide stability to the cut guide 600 while the cut is being made. In some aspects, the step 720 may include the selection of a cut slot on the cut guide 600 based on an amount of bone at the proximal end of the medial cuneiform that the physician desired to resect and remove. Once a slot (and the corresponding dimensions) has been determined, a cutting instrument (e.g., a sagittal saw, a reciprocating saw, etc.) is manipulated at least partially within a cut slot of the cut guide 610 to perform the cut. Once the cut has been completed, the step 720 may also include a removal of any bony debris between the resected surface of the medial cuneiform 204 and the first metatarsal 202.

Process 700 is shown to include a step 722 of applying fixation across the first tarsometatarsal joint, according to an exemplary embodiment. The step 722 may be performed prior to or after any/all instrument systems and components of have been decoupled from anatomy of the patient including but not limited to those shown and described herein. Step 722 may also include the incorporation of additional hardware (for example, that incorporated by reference previously herein) that is configured to facilitate the application of the fixation. The fixation may include intramedullary nails, bone plates, fasteners, or a combination of several components including but not limited to those mentioned herein. Further, step 722 may include the application of compression across the first tarsometatarsal joint so as to compress resected surfaces of the first metatarsal 202 and the medial cuneiform 204. Such compression may be achieved using the actuation instrument 410 and manipulating one or more mechanisms thereof, for example the coupling member 476 so as to bias the distal and proximal portions 444, 446 (and thus, the first metatarsal and medial cuneiform 202, 204 with which they are coupled) closer to one another along an axis substantially parallel to that of the first metatarsal 202 when in a corrected position.

Process 700 is shown to include a step 724 of decoupling the actuation instrument from the anatomy of the patient, according to an exemplary embodiment. The step 724 may include the manipulation of a locking/retention feature of the actuation instrument 410 (e.g., the actuator 478 or other components) either prior or subsequent to removal of k-wires placed in the first metatarsal 202 and the medial cuneiform 204. Step 724 may include manipulation of one or more of the coupling members 426, 460, 464, and/or 476 in order to remove and/or disassemble portions of the actuation instrument 410. The step 724 may include implementing one or more components common to closing surgical incisions, including adhesives and/or stitches/sutures/tapes. Further, this step may be performed before or after all hardware (e.g., instrumentation, systems, etc.) is removed from the patient, as this step may be performed iteratively and, as such, instrument/system/component removal may also occur iteratively.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. An alignment system, comprising:
an alignment instrument, comprising:
a lateral portion; and
a medial portion, wherein the medial portion is releasably and slidably couplable with the lateral portion;
wherein the distal portion and the medial portion are couplable with first and second bones of a patient and actuatable to apply a compression therebetween the first and second bones.

2. The alignment system of claim 1, wherein the lateral portion comprises:
an upper portion; and
a lower portion, wherein the lower portion is slidably adjustable and releasably coupled with the upper portion.

3. The alignment system of claim 2, wherein the medial portion comprises:
a proximal portion; and
a distal portion, wherein the distal portion is slidably adjustable and releasably coupled with the proximal portion.

4. The alignment system of claim 3, wherein the proximal portion is releasably couplable with a first bone of a patient and the distal portion is releasably couplable with a second bone of a patient.

5. The alignment system of claim 4, wherein the medial portion further comprises:
a first actuator threadably engaged with the proximal portion and the distal portion.

6. The alignment system of claim 5, wherein the first actuator is configured such that actuation of the first actuator increases or decreases a distance between the proximal portion and the medial portion.

7. The alignment system of claim 6, further comprising a second actuator threadably engaged with the medial portion and the lateral portion of the alignment instrument.

8. The alignment system of claim 7, wherein the second actuator is configured such that actuation of the second actuator increases or decreases a distance between the medial portion and the lateral portion.

9. The alignment system of claim 8, wherein the distal portion comprises an extension extending substantially upward from the distal portion, wherein the extension comprises:
a first prong; and
a second prong, wherein the second prong is separated from the first prong by a slot;
wherein the first and second prongs comprise a curvature comprising a consistent radius along a length thereof.

10. The alignment system of claim 9, further comprising:
a third actuator positioned at least partially within the slot, wherein the third actuator is translatable along a curved path defined by the first and second prongs.

11. The alignment system of claim 10, wherein the third actuator comprises:
a cannulation extending therethrough along a longitudinal axis from a first end to a second end; and
a locking mechanism adjustable between locked and unlocked positions, wherein the unlocked position permits translation of the third actuator within the slot and the locked position retains the third actuator at a point along the length of the slot.

12. The alignment system of claim 11, further comprising:
a positioning instrument, comprising:
a wedge-shaped geometry;
a texture disposed on an angled surface thereof; and
a handle extending from the wedge-shaped geometry.

13. The alignment system of claim 12, wherein the alignment system comprises at least one of an intramedullary nail or a plate configured to couple with the first bone and the second bone.

14. The alignment system of claim 13, wherein the proximal portion comprises:
a first opening extending from a medial surface therethrough to a lateral surface, wherein the first opening comprises a longitudinal axis having a trajectory configured to facilitate implantation of the intramedullary nail; and
a second extending from the medial surface therethrough to the lateral surface, wherein the second opening comprises a longitudinal axis having a trajectory configured to facilitate implantation of the plate.

15. An alignment system, comprising:
an alignment instrument, comprising:
a lateral portion comprising an upper portion and lower portion slidably adjustable and releasably couplable with one another;
a medial portion adjustable and releasably couplable with the lateral portion and comprising a distal portion and a proximal portion;
wherein the distal portion and the proximal portion are couplable with first and second bones of a patient and actuatable to apply a compression therebetween the first and second bones; and
a retention element releasably couplable with the upper portion of the lateral portion of the alignment instrument and configured to engage with a third bone of the patient; and
a positioning instrument.

16. The instrument system of claim 15, wherein the positioning instrument comprises a substantially wedge-shaped geometry and a texture disposed on an angled surface thereof.

17. The instrument system of claim 16, wherein the distal portion comprises an extension extending substantially upward from the distal portion, wherein the extension comprises:
a first prong; and
a second prong, wherein the second prong is separated from the first prong by a slot;
wherein the first and second prongs comprise a curvature comprising a consistent radius along a length thereof.

18. The alignment system of claim 17, further comprising:
a third actuator positioned at least partially within the slot, wherein the third actuator is translatable along a curved path defined by the first and second prongs.

19. The alignment system of claim 18, wherein the third actuator comprises:
a cannulation extending therethrough along a longitudinal axis from a first end to a second end; and
a locking mechanism adjustable between locked and unlocked positions, wherein the unlocked position permits translation of the third actuator within the slot and the locked position retains the third actuator at a point along the length of the slot.

20. A surgical method, comprising:
coupling a retention member with a first bone of a patient;
coupling a lateral portion of an alignment instrument with the retention member;
coupling a medial portion of the alignment instrument with the lateral portion of the alignment instrument;
coupling a distal aspect of the medial portion of the alignment instrument with a second bone of the patient;
coupling a proximal aspect of the medial portion of the alignment instrument with a third bone of the patient;
actuating the alignment instrument to apply compression between the second and third bones; and
applying fixation across the second and third bones.
